(19) United States
(12) Patent Application Publication
(10) Pub. No.: US 2002/0142352 A1
(43) Pub. Date: Oct. 3, 2002

(54) Novel ectoparasite salvia proteins and apparatus to collect such proteins

(75) Inventors: Shirley Wu Hunter, Ft. Collins, CO (US); Gek-Kee Sim, Fort Collins, CO (US); Eric R. Weber, Ft. Collins, CO (US)

Correspondence Address: SHERIDAN ROSS PC, 1560 BROADWAY, SUITE 1200, DENVER, CO, 80202

(73) Assignee: HESKA CORPORATION

(21) Appl. No.: 10/071,751

(22) Filed: Feb. 7, 2002

Related U.S. Application Data
(62) Division of application No. 09/171,156, filed on Mar. 4, 1999, now Pat. No. 6,368,846.
Application No. 09/171,156 is a 371 of international application No. PCT/US97/05959, filed on Apr. 10, 1997.

(51) Int. Cl. G01N 33/53; C07H 21/04; C12N 9/24; C12N 9/50; C12N 5/06; C12P 21/02

(52) U.S. Cl. 435/7.1; 435/69.1; 435/200; 435/219; 435/320.1; 435/348; 536/23.2

(57) ABSTRACT

The present invention is directed to a novel product and method for isolating ectoparasite saliva proteins, and a novel product and method for detecting and/or treating allergic dermatitis in an animal. The present invention also relates to ectoparasite saliva proteins, nucleic acid molecules having sequences that encode such proteins, and antibodies raised against such proteins. The present invention also includes methods to obtain such proteins and to use such proteins to identify animals susceptible to or having allergic dermatitis. The present invention also includes therapeutic compositions comprising such proteins and their use to treat animals susceptible to or having allergic dermatitis.

### FIELD OF THE INVENTION

The present invention relates to a novel product and method for isolating ectoparasite saliva proteins, and a novel product and method for detecting and/or treating allergic dermatitis in an animal.

### BACKGROUND OF THE INVENTION

Bites from ectoparasites, in particular fleas, can cause a hypersensitive response in animals. In particular, hypersensitive responses to fleabites is manifested in a disease called flea allergy dermatitis (FAD). Hypersensitivity refers to a state of altered reactivity in which an animal, having been previously exposed to a compound, exhibits an allergic response to the compound upon subsequent exposures. Hypersensitive responses include immediate and delayed-type hypersensitivity, and in particular Type I, Type II, Type III and Type IV hypersensitivities (described in detail in Janeway et al., Immunobiology, Garland Publishing, New York, 1994, which is incorporated in its entirety by this reference).

Foreign compounds that induce symptoms of immediate and/or delayed hypersensitivity are herein referred to as allergens. The term “allergen” primarily refers to foreign compounds capable of causing an allergic response. The term can be used interchangeably with the term “antigen,” especially with respect to a foreign compound capable of inducing symptoms of immediate and/or delayed hypersensitivity. Factors that influence an animal's susceptibility to an allergen can include a genetic component and/or environmental exposure to an allergen. Animals can be de-sensitized to an allergen by repeated injections of the allergen to which an animal is hypersensitive.

FAD can have manifestations of both immediate and delayed-type hypersensitivity (described in detail in Janeway et al., ibid.). Effective treatment of FAD has been difficult if not impossible to achieve. FAD afflicts about 15% of cats and dogs in flea endemic areas and the frequency is increasing each year. In a geographical area, effective flea control requires treatment of all animals. One treatment investigators have proposed includes desensitization of animals using flea allergens. However, reliable, defined preparations of flea allergens are needed for such treatments.

Until the discovery of the novel formulations of the present invention, flea allergens responsible for FAD had not been clearly defined. Whole flea antigen preparations have been used to diagnose and desensitize animals with FAD (Benjamini et al., 1960, pp. 214-222, Experimental Parasitology, Vol. 10; Keep et al., 1967, pp. 425-426, Australian Veterinary Journal, Vol. 43; Kristensen et al., 1978, pp. 414-423, Nord. Vet-Med, Vol. 30; Van Winkle, 1981, pp. 343-354, J. Amer. Animal Hosp. Assoc., Vol. 17; Haliwell et al., 1987, pp. 203-213, Veterinary Immunology and Immunopathology, Vol. 15; Greene et al., 1993, pp. 69-74, Parasite Immunology, Vol. 15); PCT Publication No. WO 93/18788 by Opdebeeck et al.; and Van Winkle, pp. 343-354, 1981, J. Am. Anim. Hosp. Assoc., vol. 32. Available commercial whole flea extracts, however, are unpredictable and, therefore, have limited usefulness.

Prior investigators have suggested that products contained in flea saliva might be involved in FAD and have also suggested methods to isolate such products: Benjamini et al., 1963, pp. 143-154, Experimental Parasitology, Vol. 13; Young et al., 1963, pp. 155-166, Experimental Parasitology 13, Vol. 13; Michaeli et al., 1965, pp. 162-170, J. Immunol., Vol. 95; and Michaeli et al., 1996, pp. 402-406, J. Immunol., Vol. 97. These investigators, however, have characterized the allergenic factors of flea saliva as being haptens having molecular weights of less than 6 kilodaltons (kD). That they are not proteins is also supported by the finding that they are not susceptible to degradation when exposed to strong acids (e.g., 6 N hydrochloric acid) or heat. Some of the particular low molecular weight allergenic factors have also been characterized as being a highly fluorescent aromatic fraction (Young et al., ibid.). In addition, studies by such investigators have indicated that in order to be allergenic, such factors need to be associated with adjuvants and/or carriers, such as collagen or portions of the membrane used to collect the oral secretions. Moreover, the methods described to collect flea saliva factors were difficult and unpredictable. Furthermore the factors isolated by these methods were typically contaminated with material from the fleas, their culture medium or the skin-based membranes used to allow the fleas to feed.

Thus, there remains a need to more clearly define flea saliva allergens capable of inducing a hypersensitive response in animals. In addition, there remains a need to develop a method to collect substantially pure flea saliva allergens which provide predictable and less expensive preparations of allergens useful for desensitizing animals subject to, or having, FAD.

### SUMMARY OF THE INVENTION

One embodiment of the present invention is an isolated nucleic acid molecule that hybridizes under stringent conditions with a gene including a flea saliva gene comprising a nucleic acid sequence including SEQ ID NO:52, SEQ ID NO:54, SEQ ID NO:55, SEQ ID NO:57, SEQ ID NO:58, SEQ ID NO:60, SEQ ID NO:61, SEQ ID NO:63, SEQ ID NO:64, SEQ ID NO:66, SEQ ID NO:67, SEQ ID NO:69, SEQ ID NO:71, SEQ ID NO:73, SEQ ID NO:74, SEQ ID NO:76 and a nucleic acid sequence encoding an amino acid sequence selected from the group consisting of SEQ ID NO:78 and SEQ ID NO:87.

The present invention also includes a nucleic acid molecule that hybridizes under stringent hybridization conditions with a nucleic acid molecule having a nucleic acid sequence encoding a protein comprising an amino acid sequence including SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87.

Another embodiment of the present invention includes an isolated protein encoded by a nucleic acid molecule that hybridizes under stringent hybridization conditions with a nucleic acid molecule having a nucleic acid sequence encoding a protein comprising an amino acid sequence including SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87.

Also included in the present invention are recombinant molecules and cells having a nucleic acid molecule of the present invention.

Another aspect of the present invention includes an antibody capable of selectively binding to an ectoparasite protein, or mimetope.

Yet another embodiment of the present invention is a therapeutic composition for treating allergic dermatitis comprising a formulation comprising at least one isolated ectoparasite saliva protein, wherein said ectoparasite saliva protein comprises at least a portion of an amino acid sequence, wherein said portion is encoded by a nucleic acid molecule that hybridizes under stringent hybridization conditions with a nucleic acid molecule having a nucleic acid sequence including SEQ ID NO:52, SEQ ID NO:54, SEQ ID NO:55, SEQ ID NO:57, SEQ ID NO:58, SEQ ID NO:60, SEQ ID NO:61, SEQ ID NO:63, SEQ ID NO:64, SEQ ID NO:66, SEQ ID NO:67, SEQ ID NO:69, SEQ ID NO:71, SEQ ID NO:73, SEQ ID NO:74, SEQ ID NO:76 and a nucleic acid sequence encoding an amino acid sequence selected from the group consisting of SEQ ID NO:78 and SEQ ID NO:87. A preferred therapeutic composition of the present invention also includes an excipient, an adjuvant and/or a carrier. Also included in the present invention is a method to desensitize a host animal to allergic dermatitis. The method includes the step of administering to the animal a therapeutic composition of the present invention. Other embodiments of the present invention include methods to identify an animal susceptible to or having allergic dermatitis, using in vivo or in vitro methods. In one embodiment, an animal susceptible to or having allergic dermatitis is identified in vivo by the method comprising: (a) administering to a site on the animal a formulation comprising at least one isolated ectoparasite saliva protein, in which the ectoparasite saliva protein comprises an amino acid sequence including SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87; and (b) comparing a reaction resulting from administration of the formulation with a reaction resulting from administration of a control solution, in which the animal is determined to be susceptible to or to have allergic dermatitis if the reaction to the formulation is at least as large as said reaction to the positive control solution, and in which the animal is determined not to be susceptible to or not to have allergic dermatitis if the reaction to the formulation is about the same size as said reaction to the negative control solution.

In another embodiment, an animal susceptible to or having allergic dermatitis is identified in vitro by measuring the presence of antibodies indicative of allergic dermatitis in the animal using the method comprising: (a) contacting a formulation with a body fluid from an animal under conditions sufficient for formation of an immunocomplex between the formulation and the antibodies, if present, in the body fluid, the formulation comprising at least one isolated ectoparasite saliva protein, in which the ectoparasite saliva protein comprises an amino acid sequence including SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87; and (b) determining the amount of immunocomplex formed, in which formation of the immunocomplex indicates that the animal is susceptible to or has allergic dermatitis.

The present invention further relates to an assay kit for testing if an animal is susceptible to or has allelic dermatitis, the kit comprising: (a) a formulation comprising at least one isolated ectoparasite saliva protein, in which the ectoparasite saliva protein comprises an amino acid sequence including SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87; and (b) a means for determining if the animal is susceptible to or has allergic dermatitis, in which the means comprises use of the formulation to identify animals susceptible to or having allergic dermatitis.

### DETAILED DESCRIPTION OF THE INVENTION

The present invention includes a novel product and method for diagnosing and treating allergic dermatitis of animals to ectoparasites.

According to the present invention, ectoparasites are external living parasites that attach and feed through the skin of a host animal. Ectoparasites include parasites that live on a host animal and parasites that attach temporarily to an animal in order to feed. Also, according to the present invention, ectoparasite saliva refers to the material released from the mouth of an ectoparasite when the ectoparasite attempts to feed in response to a temperature differential. Ectoparasite saliva includes ectoparasite saliva products.

One embodiment of the present invention is a formulation that contains ectoparasite saliva products that can be used to diagnose and/or treat animals susceptible to or having (i.e., suffering from) allergic dermatitis. Preferred types of allergic dermatitis to diagnose and/or treat using ectoparasite saliva products of the present invention include flea allergy dermatitis, Culicoides allergy dermatitis and mosquito allergy dermatitis. A preferred type of allergic dermatitis to diagnose and/or treat using ectoparasite saliva products of the present invention is flea allergy dermatitis. As used herein, an animal that is susceptible to allergic dermatitis refers to an animal that is genetically pre-disposed to developing allergic dermatitis and/or to an animal that has been primed with an antigen in such a manner that re-exposure to the antigen results in symptoms of allergy that can be perceived by, for example, observing the animal or measuring antibody production by the animal to the antigen. As such, animals susceptible to allergic dermatitis can include animals having sub-clinical allergic dermatitis. Sub-clinical allergic dermatitis refers to a condition in which allergy symptoms cannot be detected by simply observing an animal (i.e., manifestation of the disease can include the presence of anti-ectoparasite saliva protein antibodies within an affected animal but no dermatitis). For example, sub-clinical allergic dermatitis can be detected using in vivo or in vitro assays of the present invention, as described in detail below. Reference to animals having allergic dermatitis includes animals that do display allergy symptoms that can be detected by simply observing an animal and/or by using in vivo or in vitro assays of the present invention, as described in detail below.

One embodiment of the present invention is a formulation that includes one or more isolated ectoparasite saliva proteins. According to the present invention, an isolated protein is a protein that has been removed from its natural milieu. An isolated ectoparasite saliva protein can, for example, be obtained from its natural source, be produced using recombinant DNA technology, or be synthesized chemically. As used herein, an isolated ectoparasite saliva protein can be a full-length ectoparasite saliva protein or any homologue of such a protein, such as an ectoparasite saliva protein in which amino acids have been deleted (e.g., a truncated version of the protein, such as a peptide), inserted, inverted, substituted and/or derivatized (e.g., by glycosylation, phosphorylation, acetylation, myristylation, prenylation, palmitation, amidation and/or addition of glycosylphosphatidyl inositol). A homologue of an ectoparasite saliva protein is a protein having an amino acid sequence that is sufficiently similar to a natural ectoparasite saliva protein amino acid sequence that a nucleic acid sequence encoding the homologue is capable of hybridizing under stringent conditions to (i.e., with) a nucleic acid molecule encoding the natural ectoparasite saliva protein (i.e., the complement of a nucleic acid sequence encoding the natural ectoparasite saliva protein amino acid sequence). A nucleic acid sequence complement of any nucleic acid sequence of the present invention refers to the nucleic acid sequence of the nucleic acid strand that is complementary to (i.e., can form a complete double helix with) the strand for which the sequence is cited. It is to be noted that a double-stranded nucleic acid molecule of the present invention for which a nucleic acid sequence has been determined for one strand that represented by a SEQ ID NO also comprises a complementary strand having a sequence that is a complement of that SEQ ID NO. As such, nucleic acid molecules of the present invention, which can be either double-stranded or single-stranded, include those nucleic acid molecules that form stable hybrids under stringent hybridization conditions with either a given SEQ ID NO denoted herein and/or with the complement of that SEQ ID NO, which may or may not be denoted herein. Methods to deduce a complementary sequence are known to those skilled in the art.

As used herein, stringent hybridization conditions refer to standard hybridization conditions under which nucleic acid molecules, including oligonucleotides, are used to identify similar nucleic acid molecules. Such standard conditions are disclosed, for example, in Sambrook et al., Molecular Cloning: A Laboratory Manual, Cold Spring Harbor Labs Press, 1989; Sambrook et al., ibid., is incorporated by reference herein in its entirety. Stringent hybridization conditions typically permit isolation of nucleic acid molecules having at least about 70% nucleic acid sequence identity with the nucleic acid molecule being used to probe in the hybridization reaction. Formulae to calculate the appropriate hybridization and wash conditions to achieve hybridization permitting 30% or less mismatch of nucleotides are disclosed, for example, in Meinkoth et al., 1984, Anal. Biochem. 138, 267-284; Meinkoth et al., ibid., is incorporated by reference herein in its entirety.

The minimal size of a protein homologue of the present invention is a size sufficient to be encoded by a nucleic acid molecule capable of forming a stable hybrid with the complementary sequence of a nucleic acid molecule encoding the corresponding natural protein. As such, the size of the nucleic acid molecule encoding such a protein homologue is dependent on nucleic acid composition and percent homology between the nucleic acid molecule and complementary sequence as well as upon hybridization conditions per se (e.g., temperature, salt concentration, and formamide concentration). The minimal size of such nucleic acid molecules is typically at least about 12 to about 15 nucleotides in length if the nucleic acid molecules are GC-rich and at least about 15 to about 17 bases in length if they are AT-rich. As such, the minimal size of a nucleic acid molecule used to encode an ectoparasite saliva protein homologue of the present invention is from about 12 to about 18 nucleotides in length. There is no limit, other than a practical limit, on the maximal size of such a nucleic acid molecule in that the nucleic acid molecule can include a portion of a gene, an entire gene, or multiple genes, or portions thereof. Similarly, the minimal size of an ectoparasite saliva protein homologue of the present invention is from about 4 to about 6 amino acids in length, with preferred sizes depending on whether a full-length, multivalent (i.e., fusion protein having more than one domain each of which has a function), or functional portions of such proteins are desired.

Ectoparasite saliva protein homologues can be the result of allelic variation of a natural gene encoding an ectoparasite saliva protein. A natural gene refers to the form of the gene found most often in nature. Ectoparasite saliva protein homologues can be produced using techniques known in the art including, but not limited to, direct modifications to a gene encoding a protein using, for example, classic or recombinant DNA techniques to effect random or targeted mutagenesis.

Preferred ectoparasite saliva proteins of the present invention, including homologues thereof, are capable of detecting and/or treating allergic dermatitis resulting from the bites of ectoparasites. A preferred ectoparasite saliva protein homologue includes at least one epitope capable of eliciting a hypersensitive response to the natural ectoparasite saliva protein counterpart. An ectoparasite saliva protein homologue can also include an epitope capable of hyposensitizing an animal to the natural form of the protein. The ability of an ectoparasite saliva protein homologue to detect and/or treat (i.e., immunomodulate or regulate by, for example, desensitizing) the hypersensitivity of an animal susceptible to or having allergic dermatitis, can be tested using techniques known to those skilled in the art. Such techniques include skin tests and immunoabsorbent assays as described in detail below. Additional preferred ectoparasite saliva proteins of the present invention have other activities that include activities important for feeding and survival of the ectoparasite.

In one embodiment, a formulation of the present invention can comprise a protein having at least a portion of an isolated ectoparasite saliva protein. According to the present invention, “at least a portion of an ectoparasite saliva protein” refers to a portion of an ectoparasite saliva protein encoded by a nucleic acid molecule that is capable of hybridizing, under stringent conditions, with a nucleic acid encoding a full-length ectoparasite saliva protein of the present invention. Preferred portions of ectoparasite saliva proteins are useful for detecting and/or treating allergic dermatitis resulting from the bites of ectoparasites. Additional preferred portions have activities important for flea feeding and survival. Suitable sizes for portions of an ectoparasite saliva protein of the present invention are as disclosed for saliva protein homologues of the present invention.

As will be apparent to one of skill in the art, the present invention is intended to apply to all ectoparasites. A formulation of the present invention can include saliva products from any ectoparasites. A preferred ectoparasite of the present invention from which to isolate saliva products (including proteins), and/or from which to identify proteins that can then be produced recombinantly or synthetically, include arachnids, insects and leeches. More preferred ectoparasites from which to obtain saliva products include fleas; ticks, including both hard ticks of the family Ixodidae (e.g., Ixodes and Amblyomma) and soft ticks of the family Argasidae (e.g., Ornithodoros, such as O. parkeri and O. turicata); flies, such as midges (e.g., Culicoides), mosquitos, sand flies, black flies, horse flies, horn flies, deer flies, tsetse flies, stable flies, myiasis-causing flies and biting gnats; ants; spiders, lice; mites; and true bugs, such as bed bugs and kissing bugs, including those carrying Chagas disease. Even more preferred ectoparasite saliva products include those from fleas, mosquitos, midges, sandflies, blackflies, ticks and Rhodnius, with products from fleas, mosquitos and Culicoides being even more preferred.

A particularly preferred formulation of the present invention includes flea saliva proteins. Preferred flea saliva products include those from Ctenocephalides, Xenopsylla, Pulex, Tunga, Nosopsyllus, Diamanus, Ctopsyllus and Echidnophaga fleas, with saliva products from Ctenocephalides canis and Ctenocephalides felis fleas being even more preferred. For the purposes of illustration, many of the following embodiments discuss flea saliva proteins. Such discussion of flea saliva proteins is not intended, in any way, to limit the scope of the present invention.

In another embodiment, a formulation of the present invention includes at least a portion of an ectoparasite saliva protein homologue having at least a portion of one of the following amino acid sequences: SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87 and/or other sequences disclosed herein.

In one embodiment, a formulation of the present invention can include at least one isolated protein having (i.e., including) at least a portion of one of the amino acid sequences identified in the Sequence ID Listing, and more specifically an amino acid sequence selected from the group consisting of SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87.

It is to be appreciated that ectoparasite saliva proteins of the present invention include, but are not limited to, full-length proteins, hybrid proteins, fusion proteins, multivalent proteins, and proteins that are truncated homologues of, or are proteolytic products of, at least a portion of a protein having at least a portion of one of the following amino acid sequences: SEQ ID NO:53, SEQ ID NO:62, SEQ ID NO:65, SEQ ID NO:70, SEQ ID NO:72, SEQ ID NO:75, SEQ ID NO:77, SEQ ID NO:78, SEQ ID NO:87 and/or other sequences disclosed herein. As used herein, the term hybrid protein refers to a single protein produced from two different proteins.

The foregoing SEQ ID NO's represent amino acid sequences deduced according to methods disclosed in the Examples. It should be noted that since amino acid sequencing technology is not entirely error-free, the foregoing SEQ ID NO's, at best, represent an apparent amino acid sequence of the ectoparasite saliva proteins of the present invention. In addition, the variation seen in the foregoing SEQ ID NO's can also be due, at least in part, to allelic variation since the proteins being sequenced were derived from populations of fleas.

According to the present invention, a formulation of the present invention can include flea saliva proteins that have undergone post-translational modification. Such modification can include, for example, glycosylation. GlycQsylation can include addition of N-linked and/or O-linked oligosaccharides. It is to be appreciated that post-translational modification of a protein of the present invention can contribute to an epitope's ability to induce an allergic response against the protein in an immediate or delayed hypersensitivity response.

Another embodiment of the present invention is an isolated nucleic acid molecule capable of hybridizing, under stringent conditions, with an ectoparasite saliva protein gene encoding an ectoparasite saliva protein of the present invention. In accordance with the present invention, an isolated nucleic acid molecule is a nucleic acid molecule that has been removed from its natural milieu (i.e., that has been subject to human manipulation). As such, “isolated” does not reflect the extent to which the nucleic acid molecule has been purified. An isolated nucleic acid molecule can include DNA, RNA, or derivatives of either DNA or RNA.

An isolated nucleic acid molecule of the present invention can be obtained from its natural source either as an entire (i.e., complete) gene or a portion thereof capable of forming a stable hybrid with that gene. As used herein, the phrase “at least a portion of” an entity refers to an amount of the entity that is at least sufficient to have the functional aspects of that entity. For example, at least a portion of a nucleic acid sequence, as used herein, is an amount of a nucleic acid sequence capable of forming a stable hybrid with the corresponding gene under stringent hybridization conditions. An isolated nucleic acid molecule of the present invention can also be produced using recombinant DNA technology (e.g., polymerase chain reaction (PCR) amplification, cloning) or chemical synthesis. Isolated ectoparasite saliva protein nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications do not substantially interfere with the nucleic acid molecule's ability to encode an ectoparasite saliva protein of the present invention or to form stable hybrids under stringent conditions with natural nucleic acid molecule isolates.

An isolated nucleic acid molecule of the present invention can include a nucleic acid sequence that encodes at least one ectoparasite saliva protein of the present invention, examples of such proteins being disclosed herein. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding an ectoparasite saliva protein. As heretofore disclosed, ectoparasite saliva proteins of the present invention include, but are not limited to, proteins having full-length ectoparasite saliva protein coding regions, portions thereof, and other ectoparasite saliva protein homologues.

It is to be appreciated that an ectoparasite saliva protein of the present invention can be encoded by a full-length nucleic acid sequence which encodes a polyprotein. The polyprotein can be post-translationally processed into multiple proteins which are found in saliva. As used herein, an ectoparasite saliva protein gene includes all nucleic acid sequences related to a natural ectoparasite saliva protein gene such as regulatory regions that control production of an ectoparasite saliva protein encoded by that gene (such as, but not limited to, transcription, translation or post-translation control regions) as well as the coding region itself. A nucleic acid molecule of the present invention can be an isolated natural ectoparasite saliva protein nucleic acid molecule or a homologue thereof. A nucleic acid molecule of the present invention can include one or more regulatory regions, full-length or partial coding regions, or combinations thereof. The minimal size of an ectoparasite saliva protein nucleic acid molecule of the present invention is the minimal size capable of forming a stable hybrid under stringent hybridization conditions with a corresponding natural gene.

An ectoparasite saliva protein nucleic acid molecule homologue can be produced using a number of methods known to those skilled in the art (see, for example, Sambrook et al., ibid.). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid (e.g., the ability of a homologue to elicit an allergic response in animals having allergic dermatitis or the ability of a homologue to act as an anti-coagulant) and/or by hybridization with isolated ectoparasite saliva protein nucleic acids under stringent conditions.

One embodiment of the present invention is an ectoparasite saliva protein nucleic acid molecule that encodes a protein having at least a portion of one or more of the following amino acid sequences: SEQ ID NO:1, as well as with the complements of any of these sequences or homologues thereof. Such preferred nucleic acid molecules can hybridize to the coding and/or complementary strand.

A preferred nucleic acid molecule of the present invention is capable of hybridizing under stringent conditions to the coding strand and/or to the strand complementary to the coding strand of a nucleic acid molecule that encodes at least a portion of such a flea saliva protein or homologue thereof. A particularly preferred nucleic acid sequence is a nucleic acid sequence having at least about 65 percent, preferably at least about 75 percent, more preferably at least about 85 percent, and even more preferably at least about 95 percent homology with a nucleic acid sequence encoding at least a portion of one or more of the following amino acid sequences:SEQ ID NO:53, SEQ ID NO:62, SEQ ID NO:65, SEQ ID NO:70, SEQ ID NO:72, SEQ ID NO:75, SEQ ID NO:77, SEQ ID NO:78 and/or SEQ ID NO:87.

Such nucleic acid molecules can be a full-length gene and/or a nucleic acid molecule encoding a full-length protein, a hybrid protein, a fusion protein, a multivalent protein or a truncation fragment. More preferred nucleic acid molecules of the present invention comprise isolated nucleic acid molecules having a nucleic acid sequence as represented by SEQ ID NO:52, SEQ ID NO:54, SEQ ID NO:55, SEQ ID NO:57, SEQ ID NO:58, SEQ ID NO:60, SEQ ID NO:61, SEQ ID NO:63, SEQ ID NO:64, SEQ ID NO:66, SEQ ID NO:67, SEQ ID NO:69, SEQ ID NO:71, SEQ ID NO:73, SEQ ID NO:74, SEQ ID NO:76, a nucleic acid sequence encoding amino acid sequence SEQ ID NO:78 or SEQ ID NO:87, or other sequences disclosed herein.

SEQ ID NO:-52, a nucleic acid sequence that includes about 595 nucleotides of the apparent gene encoding flea saliva protein fspG5 (denoted nfspG5₅₉₅), encodes a protein of about 90 amino acids (denoted as PfspG5₉₀), represented by SEQ ID NO:53. The entire translation product of fspG5 is apparently about 71 amino acids and is denoted SEQ ID NO:56. SEQ ID NO:61, a nucleic acid sequence that includes about 1007 nucleotides of the apparent gene encoding flea saliva protein fspI (denoted nfspI₁₀₀₇), encodes a protein of about 155 amino acids (denoted PfspI₁₅₅), which is denoted SEQ ID NO:62. SEQ ID NO:64, a nucleic acid sequence that includes about 1205 nucleotides of the apparent gene encoding flea saliva protein fspN5 (denoted nfspN5₁₂₀₅), encodes a protein of about 353 amino acids (denoted PfspN5₃₅₃), which is denoted SEQ ID NO:65. SEQ ID NO:71, a nucleic acid sequence that includes about 406 nucleotides of the apparent gene encoding a fspN6 flea saliva protein (denoted nfspN6₄₀₆), encodes a protein of about 135 amino acids (denoted PfspN6₁₃₅), which is denoted SEQ ID NO:72. SEQ ID NO:74, a nucleic acid sequence that includes about 420 nucleotides of the apparent gene encoding a fspJ flea saliva protein, encodes a protein of about 72 amino acids, which is denoted SEQ ID NO:75.

Knowing a nucleic acid molecule of an ectoparasite saliva protein of the present invention allows one skilled in the art to make copies of that nucleic acid molecule as well as to obtain a nucleic acid molecule including additional portions of ectoparasite saliva protein-encoding genes (e.g., nucleic acid molecules that include the translation start site and/or transcription and/or translation control regions), and/or ectoparasite saliva protein nucleic acid molecule homologues. Knowing a portion of an amino acid sequence of an ectoparasite saliva protein of the present invention allows one skilled in the art to clone nucleic acid sequences encoding such an ectoparasite saliva protein. In addition, a desired ectoparasite saliva protein nucleic acid molecule can be obtained in a variety of ways including screening appropriate expression libraries with antibodies which bind to ectoparasite saliva proteins of the present invention; traditional cloning techniques using oligonucleotide probes of the present invention to screen appropriate libraries or DNA; and PCR amplification of appropriate libraries, or RNA or DNA using oligonucleotide primers of the present invention (genomic and/or cDNA libraries can be used). To isolate flea saliva protein nucleic acid molecules, preferred cDNA libraries include cDNA libraries made from unfed whole flea, fed whole flea, fed flea midgut, unfed flea midgut, and flea salivary gland. Techniques to clone and amplify genes are disclosed, for example, in Sambrook et al., ibid. The Examples section includes examples of the isolation of cDNA sequences encoding flea saliva proteins of the present invention.

The present invention also includes nucleic acid molecules that are oligonucleotides capable of hybridizing, under stringent conditions, with complementary regions of other, preferably longer, nucleic acid molecules of the present invention that encode at least a portion of one or more of the following amino acid sequences: SEQ ID NO:53, SEQ ID NO:62, SEQ ID NO:65, SEQ ID NO:70, SEQ ID NO:72, SEQ ID NO:75, SEQ ID NO:77, SEQ ID NO:78, SEQ ID NO:87, or homologues thereof, such oligonucleotides can hybridize to the coding or non-coding strand of a double-stranded nucleic acid molecule. Certain preferred oligonucleotides are capable of hybridizing to nucleic acid molecules including nucleic acid sequences represented by SEQ ID NO:52, SEQ ID NO:58, SEQ ID NO:61, SEQ ID NO:64, SEQ ID NO:71, SEQ ID NO:74, a nucleic acid sequence that encodes SEQ ID NO:78 or SEQ ID NO:87, or complements thereof.

Oligonucleotides of the present invention can be RNA, DNA, or derivatives of either. The minimal size of such oligonucleotides is the size required to form a stable hybrid between a given oligonucleotide and the complementary sequence on another nucleic acid molecule of the present invention. Minimal size characteristics are disclosed herein. The size of the oligonucleotide must also be sufficient for the use of the oligonucleotide in accordance with the present invention. Oligonucleotides of the present invention can be used in a variety of applications including, but not limited to, as probes to identify additional nucleic acid molecules, as primers to amplify or extend nucleic acid molecules or in therapeutic applications to inhibit, for example, expression of saliva proteins by ectoparasites. Such therapeutic applications include the use of such oligonucleotides in, for example, antisense-, triplex formation-, ribozyme- and/or RNA drug-based technologies. The present invention, therefore, includes such oligonucleotides and methods to interfere with the production of ectoparasite saliva proteins by use of one or more of such technologies.

The present invention also includes a recombinant vector, which includes an ectoparasite saliva protein nucleic acid molecule of the present invention inserted into any vector capable of delivering the nucleic acid molecule into a host cell. Such a vector contains heterologous nucleic acid sequences, that is nucleic acid sequences that are not naturally found adjacent to ectoparasite saliva protein nucleic acid molecules of the present invention. The vector can be either RNA or DNA, either prokaryotic or eukaryotic, and typically is a virus or a plasmid. Recombinant vectors can be used in the cloning, sequencing, and/or otherwise manipulating of ectoparasite saliva protein nucleic acid molecules of the present invention. One type of recombinant vector, herein referred to as a recombinant molecule and described in more detail below, can be used in the expression of nucleic acid molecules of the present invention. Preferred recombinant vectors are capable of replicating in the transformed cell.

A preferred nucleic acid molecule to include in a recombinant vector of the present invention is a nucleic acid molecule that encodes at least a portion of one or more of the following amino acid sequences: SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87, or other sequences disclosed herein, or homologues thereof, and nucleic acid molecules including at least a portion of a nucleic acid sequence represented by SEQ ID NO:52, SEQ ID NO:58, SEQ ID NO:61, SEQ ID NO:64, SEQ ID NO:71, SEQ ID NO:74, a nucleic acid sequence that encodes SEQ ID NO:78 or SEQ ID NO:87, or other sequences disclosed herein, or complements thereof. A more preferred sequences to include in a recombinant vector include nfspG5₅₉₅, nfspG5 ₂₇₀ nfspG5₂₁₃, nfspI1007, nfspN5₁₂₀₅, nfspN5 ₁₀₅₉ nfspN6 ₄₀₆ and nfspJ₄₂₀.

Preferred recombinant molecules of the present invention include pCro-nfspG5₂₁₃ and pCro-nfspI₄₇₄, the production of which are described in detail in the Examples section.

In one embodiment, an isolated ectoparasite saliva protein of the present invention is produced by culturing a cell capable of expressing the protein under conditions effective to produce the protein, and recovering the protein. A preferred cell to culture is a recombinant cell that is capable of expressing the ectoparasite saliva protein, the recombinant cell being produced by transforming a host cell with one or more nucleic acid molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained. Preferred nucleic acid molecules with which to transform a host cell include one or more nucleic acid molecules that are as disclosed herein for including in recombinant vectors of the present invention.

Suitable host cells to transform include any cell that can be transformed and that can express the introduced ectoparasite saliva protein. Such cells are, therefore, capable of producing ectoparasite saliva proteins of the present invention after being transformed with at least one nucleic acid molecule of the present invention. Host cells can be either untransformed cells or cells that are already transformed with at least one nucleic acid molecule. Suitable host cells of the present invention can include bacterial, fungal (including yeast), insect, animal and plant cells. Preferred host cells include bacterial, yeast, insect and mammalian cells, with bacterial (e.g., E. coli) and insect (e.g., Spodoptera) cells being particularly preferred.

A recombinant cell is preferably produced by transforming a host cell with one or more recombinant molecules, each comprising one or more nucleic acid molecules of the present invention operatively linked to an expression vector containing one or more transcription control sequences. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, insect, animal, and/or plant cells. As such, nucleic acid molecules of the present invention can be operatively linked to expression vectors containing regulatory sequences such as promoters, operators, repressors, enhancers, termination sequences, origins of replication, and other regulatory sequences that are compatible with the recombinant cell and that control the expression of nucleic acid molecules of the present invention. As used herein, a transcription control sequence includes a sequence which is capable of controlling the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, helminth, insect and mammalian cells, such as, but not limited to, tac, lac trp, trc, oxy-pro, omp/lpp, rrnB, bacteriophage lambda (λ) (such as λp_{L} and λp_{R} and fusions that include such promoters), bacteriophage T7, T71ac, bacteriophage T3, bacteriophage SP6, bacteriophage SPOL, metallothionein, alpha mating factor, Pichia alcohol oxidase, alphavirus subgenomic promoters (such as Sindbis virus subgenomic promoters), baculovirus, Heliothis zea insect virus, vaccinia virus, herpesvirus, poxyirus, adenovirus, simian virus 40, retrovirus actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences of the present invention can also include naturally occurring transcription control sequences naturally associated with a DNA sequence encoding an ectoparasite saliva protein.

Expression vectors of the present invention may also contain secretory signals (i.e., signal segment nucleic acid sequences) to enable an expressed ectoparasite saliva protein to be secreted from the cell that produces the protein. Suitable signal segments include an ectoparasite saliva protein signal segment or any heterologous signal segment capable of directing the secretion of an ectoparasite saliva protein, including fusion proteins, of the present invention. Preferred signal segments include, but are not limited to, tissue plasminogen activator (t-PA), interferon, interleukin, growth hormone, histocompatibility and viral envelope glycoprotein signal segments.

Expression vectors of the present invention may also contain fusion sequences which lead to the expression of inserted nucleic acid molecules of the present invention as fusion proteins. Inclusion of a fusion sequence as part of an ectoparasite nucleic acid molecule of the present invention can enhance the stability during production, storage and/or use of the protein encoded by the nucleic acid molecule. Furthermore, a fusion segment can function as a tool to simplify purification of an ectoparasite saliva protein, such as to enable purification of the resultant fusion protein using affinity chromatography. A suitable fusion segment can be a domain of any size that has the desired function (e.g., increased stability and/or purification tool). It is within the scope of the present invention to use one or more fusion segments. Fusion segments can be joined to amino and/or carboxyl termini of an ectoparasite saliva protein. Linkages between fusion segments and ectcparasite saliva proteins can be constructed to be susceptible to cleavage to enable straight-forward recovery of the ectoparasite saliva proteins. Fusion proteins are preferably produced by culturing a recombinant cell transformed with a fusion nucleic acid sequence that encodes a protein including the fusion segment attached to either the carboxyl and/or amino terminal end of an ectoparasite saliva protein.

A recombinant molecule of the present invention is a molecule that can include at least one of any nucleic acid molecule heretofore described operatively linked to at least one of any transcription control sequence capable of effectalveoli regulating expression of the nucleic acid molecule(s) in the cell to be transformed. A preferred recombinant molecule includes one or more nucleic acid molecules that are as disclosed herein for including in a recombinant vector of the present invention.

A recombinant cell of the present invention includes any cells transformed with at least one of any nucleic acid molecules of the present invention. A preferred recombinant cell is a cell transformed with at least one nucleic acid molecule that encode a protein having at least a portion of one or more of the following amino acid sequences: SEQ ID NO:53, SEQ ID NO:62, SEQ ID NO:65, SEQ ID NO:70, SEQ ID NO:72, SEQ ID NO:75, SEQ ID NO:77, SEQ ID NO:78, SEQ ID NO:87, or other sequences disclosed herein, or homologues thereof, and nucleic acid molecules including at least a portion of a nucleic acid sequence represented by SEQ ID NO:52, SEQ ID NO:58, SEQ ID NO:61, SEQ ID NO:64, SEQ ID NO:71, SEQ ID NO:74, a nucleic acid sequence that encodes SEQ ID NO:78 or SEQ ID NO:87, or other sequences disclosed herein, or complements thereof. Particularly preferred recombinant cells include E. coli transformed with at least one of the aforementioned nucleic acid molecules. Preferred recombinant cells of the present invention include E. coli:pCro-nfspG5₂₁₃ and E. coli:pCro-nfspI₄₇₄.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant protein production during fermentation. The activity of an expressed recombinant protein of the present invention may be improved by fragmenting, modifying, or derivatizing the resultant protein.

In accordance with the present invention, recombinant cells can be used to produce an ectoparasite saliva protein of the present invention by culturing such cells under conditions effective to produce such a protein, and recovering the protein. Effective conditions to produce a protein include, but are not limited to, appropriate media, bioreactor, temperature, pH and oxygen conditions that permit protein production. An appropriate, or effective, medium refers to any medium in which a cell of the present invention, when cultured, is capable of producing an ectoparasite saliva protein. Such a medium is typically an aqueous medium comprising assimilable carbohydrate, nitrogen and phosphate sources, as well as appropriate salts, minerals, metals and other nutrients, such as vitamins. The medium may comprise complex nutrients or may be a defined minimal medium.

Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant ectoparasite saliva proteins may either remain within the recombinant cell; be secreted into the fermentation medium; be secreted into a space between two cellular membranes, such as the periplasmic space in E. coli; or be retained on the outer surface of a cell or viral membrane. The phrase “recovering the protein” refers simply to collecting the whole fermentation medium containing the protein and need not imply additional steps of separation or purification. Ectoparasite saliva proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, chromatofocusing and differential solubilization.

Ectoparasite saliva proteins are preferably retrieved in “substantially pure” form. As used herein, “substantially pure” refers to a purity that allows for the effective use of the protein as a therapeutic composition or diagnostic. For example, an animal being administered dosages of ectoparasite saliva protein isolated from a recombinant cell of the present invention should exhibit no substantial toxicity from contaminants mixed with the protein.

Ectoparasite saliva that is substantially free of contaminating material can be collected using a saliva collection apparatus of the present invention (disclosed in related PCT Patent Publication No. WO 96/11,271, published Apr. 18, 1996, by Frank et al.; this publication is incorporated by reference herein in its entirety). The interior diameter of a preferred chamber of the present invention is preferably about 7.5 cm. The size of a collection means of the present invention is preferably larger than the open end of the 7.5 cm chamber, the size of the collection means is more preferably about 8 cm.

According to the present invention, ectoparasite saliva products can be extracted from a collection means (described in related PCT Patent Publication No. WO 96/11,271) by contacting a collection means with a Tris buffer containing sodium chloride, alcohol and Tris. A more preferred extraction buffer includes 2.5 M NaCl, 5% IPA and 20 mM Tris, about pH 8.0 to about pH 8.3. Suitable extraction times for eluting proteins and other products from the collection means using the Tris buffer are described in detail in the Examples.

Further concentration of saliva proteins extracted from a collection means of the present invention can be performed by concentrating the extracted flea saliva product-containing solution using hydrophobic interaction chromatographic (HIC) resins. Suitable HIC resins include any resins that bind protein at high salt concentrations. Preferred HIC resins include, for example, butyl-, octyl- and phenyl-substrate conjugated resins. A more preferred resin includes a phenyl-sepharose resin. In a preferred embodiment, extracted flea saliva proteins contained in a Tris buffer of the present invention can be contacted with a HIC resin to bind the flea saliva proteins to the resin.

In accordance with the present invention, a “mimetope” refers to any compound that is able to mimic the ability of an isolated ectoparasite saliva protein of the present invention to carry out its function (e.g., anti-coagulation, anti-complement, vasodialators, proteases, acid phosphatases or detecting and/or treating the hypersensitivity of an animal susceptible to or having allergic dermatitis). A mimetope can be a peptide that has been modified to decrease its susceptibility to degradation but that still retains the desired activity. Other examples of mimetopes include, but are not limited to, carbohydrate-based compounds, lipid-based compounds, nucleic acid-based compounds, natural organic compounds, synthetically derived organic compounds, anti-idiotypic antibodies and/or catalytic antibodies, or fragments thereof. Mimetopes of the present invention can also include non-proteinaceous portions of ectoparasite saliva products having allergenic and/or antigenic activity (e.g., carbohydrate moieties associated with ectoparasite saliva proteins). A mimetope can be obtained by, for example, screening libraries of synthetic compounds for compounds capable of altering the ability of ectoparasites to feed, or of detecting and/or treating allergic dermatitis resulting from the bites of ectoparasites. A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modeling. The predicted mimetope structures can then be produced by, for example, chemical synthesis, recombinant DNA technology, or by isolating a mimetope from a natural source (e.g., plants, animals, bacteria and fungi).

One embodiment of the present invention is an in vivo test that is capable of detecting whether an animal is hypersensitive to ectoparasite saliva products. An in vivo test of the present invention can initially be used to determine if an animal is hypersensitive to ectoparasite saliva products and then used to determine if an animal is hypersensitive to a particular ectoparasite saliva component, in particular to an ectoparasite saliva protein. An in vivo hypersensitivity test of the present invention is particularly useful for identifying animals susceptible to or having allergic dermatitis. An in vivo hypersensitivity test of the present invention is even more useful for identifying animals susceptible to or having FAD. A suitable in vivo hypersensitivity test of the present invention can be, but is not limited to, a skin test comprising administering (e.g., intradermally injecting or superficial scratching) an effective amount of a formulation containing at least one ectoparasite saliva product, or a mimetope thereof. Methods to conduct skin tests of the present invention are known to those of skill in the art and are briefly disclosed herein.

Suitable formulations to use in an in vivo skin test include one or more isolated ectoparasite saliva proteins of the present invention.

A suitable amount of ectoparasite saliva protein for use in a skin test of the present invention can vary widely depending on the allergenicity of the product used in the test and on the site at which the product is delivered. Suitable amounts of ectoparasite saliva proteins for use in a skin test of the present invention include an amount capable of forming reaction, such as a detectable wheal or induration (hardness) resulting from an allergic reaction to the product. Preferred amounts of ectoparasite saliva proteins for use in a skin test of the present invention range from about 1 nanogram (ng) to about 500 micrograms (ug), more preferably from about 5 ng to about 300 ug, and even more preferably from about 10 ng to about 50 ug of ectoparasite saliva proteins. It is to be appreciated by those of skill in the art that such amounts will vary depending upon the allergenicity of the protein(s) being administered.

According to the present invention, ectoparasite saliva proteins of the present invention can be combined with an immunopotentiator (e.g., carriers or adjuvants of the present invention as defined in detail below). A novel aspect, however, of the present invention is that an ectoparasite saliva protein of the present invention can induce a hypersensitive response in the absence of an immunopotentiator.

A skin test of the present invention further comprises administering a control solution to an animal. A control solution can include a negative control solution and/or a positive control solution. A positive control solution of the present invention contains an effective amount of at least one compound known to induce a hypersensitive response when administered to an animal. A preferred compound for use as positive control solution includes, but is not limited to, histamine. A negative control solution of the present invention can comprise a solution that is known not to induce a hypersensitive response when administered to an animal. As such, a negative control solution can comprise a solution having compounds essentially incapable of inducing a hypersensitive response or simply a buffer used to prepare the formulation, such as saline. An example of a preferred negative control solution is phenolated phosphate buffered saline (available from Greer Laboratories, Inc., Lenoir, N.C.).

Hypersensitivity of an animal to one or more formulations of the present invention can be evaluated by measuring reactions (e.g., wheal size, induration or hardness; using techniques known to those skilled in the art) resulting from administration of one or more experimental sample(s) and control sample(s) into an animal and comparing the reactions to the experimental sample(s) with reactions resulting from administration of one or more control solution. Preferred devices for intradermal injections include individual syringes. Preferred devices for scratching include devices that permit the administration of a number of samples at one time. The hypersensitivity of an animal can be evaluated by determining if the reaction resulting from administration of a formulation of the present invention is larger than the reaction resulting from administration of a negative control, and/or by determining if the reaction resulting from administration of the formulation is at least about the same size as the reaction resulting from administration of a positive control solution. As such, if an experimental sample produces a reaction greater than or equal to the size of a wheal produced by administration of a positive control sample to an animal, then that animal is hypersensitive to the experimental sample. Conversely, if an experimental sample produces a reaction similar to the reaction produced by administration of a negative control sample to an animal, then that animal is not hypersensitive to the experimental sample.

Preferred wheal sizes for evaluation of the hypersensitivity of an animal range from about 16 mm to about 8 mm, more preferably from about 15 mm to about 9 mm, and even more preferably from about 14 mm to about 10 mm in diameter.

Preferably, the ability or inability of an animal to exhibit an immediate hypersensitive response to a formulation of the present invention is determined by measuring wheal sizes from about 2 minutes to about 30 minutes after administration of a sample, more preferably from about 10 minutes to about 25 minutes after administration of a sample, and even more preferably about 15 minutes after administration of a sample.

Preferably, the ability or inability of an animal to exhibit a delayed hypersensitive response to a formulation of the present invention is determined by measuring induration and/or erythema from about 18 hours to about 30 hours after administration of a sample, more preferably from about 20 hours to about 28 hours after administration of a sample, and even more preferably at about 24 hours after administration of a sample. A delayed hypersensitivity response can also be measured using other techniques such as by determining, using techniques known to those of skill in the art, the extent of cell infiltrate at the site of administration during the time periods defined directly above.

In a preferred embodiment, a skin test of the present invention comprises intradermally injecting into an animal at a given site an effective amount of a formulation that includes at least one flea saliva protein of the present invention, and intradermally injecting an effective amount of a control solution into the same animal at a different site. It is within the scope of one of skill in the art to use devices capable of delivering multiple samples simultaneously at a number of sites, preferably enabling concurrent evaluation of numerous formulations. One preferred formulation comprises flea saliva products collected in accordance with the present invention. Also preferred are formulations comprising one or more recombinantly produced flea saliva proteins.

Suitable flea saliva proteins for use with a skin test of the present invention include proteins having an amino acid sequence such as is listed in the Sequence Listing herein, or homologues thereof. A preferred positive control sample can be a sample comprising histamine. A preferred negative control sample can be a sample comprising diluent.

Animals suitable and preferred to test for hypersensitivity to ectoparasite saliva proteins using a skin test of the present invention are disclosed herein. Particularly preferred animals to test with a skin test of the present invention include dogs, cats and horses, with dogs and cats being even more preferred.

Another embodiment of the present invention is an in vitro immunoabsorbent test that is capable of detecting the presence of an antibody capable of binding to one or more ectoparasite saliva proteins of the present invention by contacting a putative antibody-containing solution with a solution containing ectoparasite saliva proteins in such a manner that immunocomplexes can form and be detected. Thus, an in vitro immunoabsorbent test of the present invention is particularly useful for identifying animals susceptible to or having allergic dermatitis by demonstrating that an animal has been previously exposed to an ectoparasite saliva antigen and, therefore may be hypersensitive to further exposure to an ectoparasite saliva antigen.

According to the present invention, an in vitro hypersensitivity test of the present invention can be, but is not limited to, an immunoabsorbent test comprising: (a) contacting a formulation of the present invention with a body fluid from an animal under conditions sufficient for formation of an immunocomplex between the formulation and antibodies, if present, in the body fluid; and (b) determining the amount of immunocomplex formed, wherein formation of the immunocomplex indicates that the animal is susceptible to or has allergic dermatitis. The immunoabsorbent test is particularly useful for the detection of IgE antibodies in the body fluid, thereby indicating immediate hypersensitivity in the animal. Determining the amount of immunocomplex formed can include the step of separating depending on the mode of detection. Immunoabsorbent assays can be a variety of protocols and can be set-up by those of skill in the art.

A preferred immunoabsorbent test of the present invention comprises a first step of coating one or more portions of a solid substrate with a suitable amount of one or more ectoparasite saliva proteins of the present invention or a mimetope thereof, and of coating one or more other portions of the (or another) solid substrate with a suitable amount of positive and/or negative control solutions of the present invention. A preferred solid substrate of the present invention can include, but is not limited to, an ELISA plate, a dipstick, a radioimmunoassay plate, agarose beads, plastic beads, immunoblot membranes and paper; a more preferred solid substrate includes an ELISA plate, a dipstick or a radioimmunoassay plate, with an ELISA plate and a dipstick being even more preferred. As used herein, a dipstick refers to any solid material having a surface to which antibodies can be bound, such solid material having a stick-like shape capable if being inserted into a test tube. Suitable and preferred flea saliva proteins for use with an in vitro hypersensitivity test of the present invention are as disclosed for a skin test of the present invention.

A second step of a preferred in vitro hypersensitivity test of the present invention comprises contacting the coated substrate with a body fluid, such as serum, plasma or whole blood, from an animal susceptible to allergic dermatitis in such a manner as to allow antibodies contained in the body fluid that are capable of binding to ectoparasite saliva products to bind to such products bound to the substrate to form immunocomplexes. Excess body fluid and antibodies are then washed from the substrate. In a preferred embodiment in which IgE antibodies in the body fluid are to be measured, the body fluid can be pretreated to remove at least some of the other isotypes of immunoglobulin and/or other proteins, such as albumin, present in the fluid. Such removal can include, but is not limited to, contacting the body fluid with a material, such a Protein G, to remove IgG antibodies and/or affinity purifying the IgE antibodies from other components of the body fluid by exposing the fluid to, for example, Concanavalin A (Con-A).

A third step of a preferred in vitro hypersensitivity test of the present invention comprises contacting the immunocomplexes bound to the substrate with a compound capable of binding to the immunocomplexes, such as a secondary antibody or other compound that is capable of binding to the heavy chain of allergy-related antibodies produced by animals allergic to ectoparasites, in such a manner that the compound(s) can bind to the immunocomplexes. Preferred binding compounds include, but are not limited to, secondary antibodies capable of binding to the heavy chain of IgE antibodies and Fc receptors (FcR) that bind to IgE antibodies (i.e., epsilon FcR), including single chains of an FcR (e.g., the alpha chain of an epsilon FcR), as well as truncated forms with or without transmembrane domains. Preferred animals to test are disclosed herein. Compounds capable of binding to immunocomplexes are usually tagged with a label which enables the amount of compound bound to the antibody from the body fluid to be measured. Such labels include, but are not limited to, a radioactive label, an enzyme capable of producing a color reaction upon contact with a substrate, a fluorescent label, a chemiluminescent label, a chromophoric label or a compound capable of being bound by another compound. Preferred labels include, but are not limited to, fluorescein, radioisotopes, alkaline phosphatases, biotin, avidin, or peroxidases.

A fourth step of a preferred in vitro hypersensitivity test of the present invention comprises measuring the amount of detectable label bound to the solid substrate using techniques known to those of skill in the art. It is within the scope of the present invention that the amount of antibody from the body fluid bound to the substrate can be determined using one or more layers of secondary antibodies or other binding compounds. For example, an untagged secondary antibody can be bound to a serum antibody and the untagged secondary antibody can then be bound by a tagged tertiary antibody.

A hypersensitive animal is identified by comparing the level of immunocomplex formation using samples of body fluid with the level of immunocomplex formation using control samples. An immunocomplex refers to a complex comprising an antibody and its ligand (i.e., antigen). As such, immunocomplexes form using positive control samples and do not form using negative control samples. As such, if a body fluid sample results in immunocomplex formation greater than or equal to immunocomplex formation using a positive control sample, then the animal from which the fluid was taken is hypersensitive to the ectoparasite saliva product bound to the substrate. Conversely, if a body fluid sample results in immunocomplex formation similar to immunocomplex formation using a negative control sample, then the animal from which the fluid was taken is not hypersensitive to the ectoparasite saliva product bound to the substrate.

A preferred embodiment of an in vitro hypersensitivity test of the present invention comprises the steps of: (a) contacting an ELISA plate, which is coated with a suitable amount of flea saliva extract (disclosed in related PCT Patent Publication No. WO 96/11,271, published Apr. 18, 1996, by Frank et al.; this publication is incorporated by reference herein in its entirety), including FS-1, FS-2, FS-3 and/or one or more flea saliva proteins (disclosed in related PCT Patent Publication No. WO 96/11,271 and disclosed herein), with serum, plasma or whole blood from an animal being tested for susceptibility to allergic dermatitis; and (b) identifying whether immunocomplexes are formed by step (a) by assaying for the presence of such immunocomplexes by (i) contacting the plate with an antibody that specifically binds to IgE or other compounds capable of binding to such immunocomplexes, such as an epsilon Fc receptor, and (ii) determining whether such an antibody or other compound is bound thereto. It should be noted that citing of specific embodiments does not preclude the use of a variety of other immunoassay protocols, including those in which a compound that binds IgE is coated onto a substrate; the substrate is then contacted with serum, plasma or whole blood; and binding of IgE by the compound is detected by the ability to bind flea saliva extracts or proteins of the present invention.

One embodiment of the present invention is a kit useful for identification of an animal susceptible to or having allergic dermatitis. As used herein, a suspect animal is an animal to be tested. A kit of the present invention comprises a formulation of the present invention and a means for determining if an animal is susceptible to or has allergic dermatitis, in which the formulation is used to identify animals susceptible to or having allergic dermatitis. A means for determining if an animal is susceptible to or has allergic dermatitis can include an in vivo or in vitro hypersensitivity test of the present invention as described in detail above. A kit of the present invention further comprises at least one control solution such as those disclosed herein.

A preferred kit of the present invention comprises the elements useful for performing an immunoassay. A kit of the present invention can comprise one or more experimental samples (i.e., formulations of the present invention) and one or more control samples bound to at least one pre-packed dipstick or ELISA plate, and the necessary means for detecting immunocomplex formation (e.g., labeled secondary antibodies or other binding compounds and any necessary solutions needed to resolve such labels, as described in detail above) between antibodies contained in the bodily fluid of the animal being tested and the proteins bound to the dipstick or ELISA plate. It is within the scope of the invention that the kit can comprise simply a formulation of the present invention and that the detecting means can be provided in another way.

An alternative preferred kit of the present invention comprises elements useful for performing a skin test. A kit of the present invention can comprise at least one pre-packed syringe and needle apparatus containing one or more experimental samples and/or one or more control samples.

It is within the scope of the present invention that two or more different in vivo and/or in vitro tests can be used in combination for diagnostic purposes. For example, the immediate hypersensitivity of an animal to an ectoparasite saliva allergen can be tested using an in vitro immunoabsorbent test capable of detecting IgE antibodies specific for an ectoparasite saliva allergen in the animal's bodily fluid. While most animals that display delayed hypersensitivity to an ectoparasite saliva allergen also display immediate hypersensitivity to the allergen, a small number of animals that display delayed hypersensitivity to an allergen do not display immediate hypersensitivity to the allergen. In such cases, following negative results from the IgE-specific in vitro test, the delayed hypersensitivity of the animal to an ectoparasite saliva allergen can be tested using an in vivo test of the present invention.

Another aspect of the present invention includes treating animals susceptible to or having allergic dermatitis, with a formulation of the present invention. According to the present invention, the term treatment can refer to the regulation of a hypersensitive response by an animal to bites from ectoparasites. Regulation can include, for example, immunomodulation of cells involved in the animal's hypersensitive response or alteration of the ability of an ectoparasite to introduce allergens into an animal, for example by inhibiting the anti-coagulation activity of a saliva enzyme, thereby impairing the ability of the arthropod to penetrate the dermis of an animal and feed. Immunomodulation can include modulating the activity of molecules typically involved in an immune response (e.g., antibodies, antigens, major histocompatibility molecules (MHC) and molecules co-reactive with MHC molecules). In particular, immunomodulation refers to modulation of antigen:antibody interactions resulting in inflammatory responses, immunosuppression, and immunotolerization of cells involved in a hypersensitive response. Immunosuppression refers to inhibiting an immune response by, for example, killing particular cells involved in the immune response. Immunotolerization refers to inhibiting an immune response by anergizing (i.e., diminishing reactivity of a T cell to an antigen) particular cells involved in the immune response. Suitable and preferred ectoparasites against which to treat an animal are disclosed herein. A particularly preferred formulation of the present invention is used to treat FAD.

One embodiment of the present invention is a therapeutic composition that, when administered to an animal in an effective manner, is useful for immunomodulating the immune response of the animal (i.e., immunomodulating the animal) so as to block (i.e., to inhibit, reduce or substantially prevent) a hypersensitive response by the animal upon subsequent exposure to allergenic components transmitted through bites from ectoparasites. Such a therapeutic composition is useful for immunomodulating animals known to be hypersensitive to ectoparasite saliva products and animals susceptible to hypersensitive responses against ectoparasite saliva products.

One embodiment of the present invention is a therapeutic composition that includes de-sensitizing compounds capable of inhibiting an immune response to an ectoparasite saliva protein of the present invention. Such de-sensitizing compounds include blocking compounds, toleragens and/or suppressor compounds. Blocking compounds comprise compounds capable of modulating antigen:antibody interactions that can result in inflammatory responses, toleragens are compounds capable of immunotolerizing an animal, and suppressor compounds are capable of immunosuppressing an animal. A de-sensitizing compound of the present invention can be soluble or membrane-bound. Membrane-bound de-sensitizing compounds can be associated with biomembranes, including cells, liposomes, planar membranes, cochleates or micelles. A soluble de-sensitizing compound of the present invention is useful for: (1) inhibiting a Type I hypersensitivity reaction by blocking IgE:antigen mediated de-granulation of mast cells; (2) inhibiting a Type III hypersensitivity reaction by blocking IgG:antigen complex formation leading to complement destruction of cells; and (3) inhibiting a Type IV hypersensitivity reaction by blocking T helper cell stimulation of cytokine secretion by macrophages. A membrane-bound de-sensitizing compound of the present invention is useful for: (1) inhibiting a Type II hypersensitivity reaction by blocking IgG:antigen complex formation on the surface of cells leading to complement destruction of cells; (2) inhibiting a Type II hypersensitivity reaction by blocking IgG regulated signal transduction in immune cells; and (3) inhibiting a Type IV hypersensitivity reaction by blocking T cytotoxic cell killing of antigen-bearing cells.

A de-sensitizing compound of the present invention can also be covalently linked to a ligand molecule capable of targeting the de-sensitizing compound to a specific cell involved in a hypersensitive response to ectoparasite saliva products. Appropriate ligands with which to link a de-sensitizing compound include, for example, at least a portion of an immunoglobulin molecule, cytokines, lectins, heterologous allergens, CD8 molecules, CD4 molecules or major histocompatibility molecules (e.g., MHC class I or MHC class II molecules). Preferred portions of immunoglobulin molecules to link to a de-sensitizing compound include variable regions capable of binding to immune cell specific surface molecules and constant regions capable of binding to Fc receptors on immune cells, in particular IgE constant regions. Preferred CD8 molecules include at least the extracellular functional domains of the β chain of CD8. Preferred CD4 molecules include at least the extracellular functional domains of CD4. An immune cell refers to a cell involved in an immune response, in particular, cells having MHC class I or MHC class II molecules. Preferred immune cells include antigen presenting cells, T cells and B cells.

In one embodiment, a therapeutic composition of the present invention includes ectoparasite saliva products of the present invention, or mimetopes thereof. Preferred therapeutic compositions include formulations comprising ectoparasite saliva extracts or at least one ectoparasite saliva product (preferably protein) of the present invention or mimetopes thereof.

Suitable therapeutic compositions of the present invention for treating flea allergy dermatitis include flea saliva extracts (such as those disclosed in related PCT Patent Publication No. WO 96/11,271) and other formulations including at least one flea saliva protein, or a mimetope thereof. Preferred therapeutic compositions include FS-1, FS-2 and/or FS-3 (such as those disclosed in related PCT Patent Publication No. WO 96/11,271) as well as at least a portion of at least one flea saliva protein that can be isolated from FS-1, FS-2 and/or FS-3. As such, preferred formulations for use as therapeutic compositions include FS-1, FS-2, FS-3, and/or at least a portion of one or more of the proteins having an amino acid sequence including SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87

In another embodiment, a therapeutic composition can include ectoparasite products of the present invention associated with a suitable excipient. A therapeutic composition of the present invention can be formulated in an excipient that the animal to be treated can tolerate. Preferred excipients are capable of maintaining a product of the present invention in a form that is capable of being bound by cells involved in an allergic response in an animal such that the cells are stimulated to initiate or enhance an immune response. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In another embodiment, a therapeutic composition of the present invention can also comprise a carrier or adjuvant, although it is to be appreciated that an advantage of saliva products of the present invention is that adjuvants and/or carriers are not required for administration. Adjuvants are typically substances that generally enhance the immune response of an animal to a specific antigen. Suitable adjuvants include, but are not limited to, cytokines, chemokines, and compounds that induce the production of cytokines and chemokines (e.g., granulocyte macrophage colony stimulating factor [GM-CSF], macrophage colony stimulating factor [M-CSF], granulocyte colony stimulating factor [G-CSF], colony stimulating factor [CSF], erythropoietin [EPO], interleukin-2 [IL-2], interleukin-3 [IL-3], interleukin-5 [IL-5], interleukin-6 [IL-6], interleukin-7 [IL-7], interleukin-8 [IL-8], interleukin-10 [IL-10], interleukin-12 [IL-12], gamma interferon [IFN-γ], interferon gamma inducing factor [IGIF], transforming growth factor beta, RANTES [regulated upon activation, normal T cell expressed and presumably secreted], macrophage inflammatory proteins [e.g., MIP1α and MIP1β], and Leishmania elongation initiating factor [LeIF]; bacterial components (e.g., endotoxins, in particular superantigens, exotoxins and cell wall components); aluminum-based salts; calcium-based salts; silica; polynucleotides; toxoids; serum proteins, viral coat proteins; block copolymer adjuvants (e.g., HunterTs Titermax™ adjuvant [Vaxcel™, Inc. Norcross, Ga.], Ribi adjuvants [Ribi ImmunoChem Research, Inc., Hamilton, Mont.]; and saponins and their derivatives (e.g., Quil A [Superfos Biosector A/S, Denmark]. Protein adjuvants of the present invention can be delivered in the form of the protein themselves or of nucleic acid molecules encoding such proteins using the methods described herein.

Carriers are typically compounds that increase the half-life of a therapeutic composition in the treated animal. Suitable carriers include, but are not limited to, polymeric controlled release formulations, biodegradable implants, liposomes, bacteria, viruses, oils, esters, and glycols.

One embodiment of the present invention is a controlled release formulation that is capable of slowly releasing a therapeutic composition of the present invention into the bloodstream of an animal. Suitable controlled release formulations include, but are not limited to, biocompatible (including biodegradable) polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ.

The present invention also includes a recombinant virus particle therapeutic composition. Such a composition includes a recombinant molecule of the present invention that is packaged in a viral coat and that can be expressed in an animal after administration. Preferably, the recombinant molecule is packaging-deficient. A number of recombinant virus particles can be used, including, but not limited to, those based on alphaviruses, poxyiruses, adenoviruses, herpesviruses, and retroviruses. Preferred recombinant particle viruses are those based on alphaviruses (such as Sindbis virus), herpesviruses and poxyiruses. Methods to produce and use recombinant virus particle vaccines are disclosed in U.S. patent application Ser. No. 08/015/414, filed Feb. 8, 1993, entitled “Recombinant Virus Particle Vaccines”, U.S. Pat. No. 5,266,313, by Esposito et al., issued Nov. 30, 1993 and U.S. patent application Ser. No. 08/602,010, by Haanes et al., filed Jan. 15, 1996, entitled “Recombinant Canine Herpesvirus”, each of the patents and patent application referred to in this section is incorporated by reference herein in its entirety.

When administered to an animal, a recombinant virus particle therapeutic composition of the present invention infects cells within the immunized animal and directs the production of a protective protein or RNA nucleic acid molecule that is capable of protecting the animal from allergic dermatitis caused by the bites of ectoparasites. For example, a recombinant virus particle comprising a nucleic acid molecule encoding one or more ectoparasite saliva protein of the present invention is administered according to a protocol that results in the tolerization of an animal against ectoparasite saliva allergens.

According to one embodiment, a nucleic acid molecule of the present invention can be delivered to an animal as a naked (i.e., not packaged in a viral coat or cellular membrane) nucleic acid vaccine (e.g., as naked DNA or RNA molecules, such as is taught, for example in Wolff et al., 1990, Science 247, 1465-1468). A naked nucleic acid vaccine of the present invention includes a nucleic acid molecule of the present invention and preferably includes a recombinant molecule of the present invention that preferably is replication, or otherwise amplification, competent. A naked nucleic acid vaccine of the present invention can comprise one or more nucleic acid molecules of the present invention in the form of, for example, a dicistronic recombinant molecule. Preferred naked nucleic acid vaccines include at least a portion of a viral genome (i.e., a viral vector). Preferred viral vectors include those based on alphaviruses, poxyiruses, adenoviruses, herpesviruses, and retroviruses, with those based on alphaviruses (such as Sindbis or Semliki virus), species-specific herpesviruses and species-specific poxyiruses being particularly preferred. Any suitable transcription control sequence can be used, including those disclosed as suitable for protein production. Particularly preferred transcription control sequence include cytomegalovirus intermediate early (preferably in conjunction with Intron-A), Rous Sarcoma Virus long terminal repeat, and tissue-specific transcription control sequences, as well as transcription control sequences endogenous to viral vectors if viral vectors are used. The incorporation of “strong” poly(A) sequences are also preferred.

Naked nucleic acid vaccines of the present invention can be administered in a variety of ways, with intramuscular, subcutaneous, intradermal, transdermal, intranasal and oral routes of administration being preferred. An example of one embodiment is disclosed in PCT Patent Publication No. WO 95/05853, published Mar. 2, 1995. A preferred single dose of a naked nucleic acid vaccine ranges from about 1 nanogram (ng) to about 100 μg, depending on the route of administration and/or method of delivery, as can be determined by those skilled in the art. Suitable delivery methods include, for example, by injection, as drops, aerosolized, oral and/or topical. Naked DNA of the present invention can be contained in an aqueous excipient (e.g., phosphate buffered saline) alone or a carrier (e.g., lipid-based vehicles).

Therapeutic compositions of the present invention can be sterilized by conventional methods which do not result in protein degradation (e.g., filtration) and/or lyophilized.

A therapeutic composition of the present invention can be administered to any animal susceptible to ectoparasite infestation as herein described. Acceptable protocols by which to administer therapeutic compositions of the present invention in an effective manner can vary according to individual dose size, number of doses, frequency of dose administration, and mode of administration. Determination of such protocols can be accomplished by those skilled in the art. An effective dose refers to a dose capable of treating an animal against hypersensitivity to ectoparasite saliva allergens. Effective doses can vary depending upon, for example, the therapeutic composition used, the arthropod from which the composition was derived, and the size and type of the recipient animal. Effective doses to immunomodulate an animal against ectoparasite saliva allergens include doses administered over time that are capable of alleviating a hypersensitive response by an animal to ectoparasite saliva allergens. For example, a first tolerizing dose can comprise an amount of a therapeutic composition of the present invention that causes a minimal hypersensitive response when administered to a hypersensitive animal. A second tolerizing dose can comprise a greater amount of the same therapeutic composition than the first dose. Effective tolerizing doses can comprise increasing concentrations of the therapeutic composition necessary to tolerize an animal such that the animal does not have a hypersensitive response to the bite of an ectoparasite. An effective dose to desensitize an animal can comprise a concentration of a therapeutic composition of the present invention sufficient to block an animal from having a hypersensitive response to the bite of an ectoparasite. Effective desensitizing doses can include repeated doses having concentrations of a therapeutic composition that cause a minimal hypersensitive response when administered to a hypersensitive animal.

A suitable single dose is a dose that is capable of treating an animal against hypersensitivity to ectoparasite saliva allergens when administered one or more times over a suitable time period. For example, a preferred single dose of an ectoparasite saliva product, or mimetope therapeutic composition is from about 0.5 ng to about 1 g of the therapeutic composition per kilogram body weight of the animal. Further treatments with the therapeutic composition can be administered from about 1 hour to 1 year after the original administration. Further treatments with the therapeutic composition preferably are administered when the animal is no longer protected from hypersensitive responses to ectoparasite. Particular administration doses and schedules can be developed by one of skill in the art based upon the parameters discussed above. Modes of administration can include, but are not limited to, subcutaneous, intradermal, intravenous, nasal, oral, transdermal and intramuscular routes.

A therapeutic composition of the present invention can be used in conjunction with other compounds capable of modifying an animal's hypersensitivity to ectoparasite bites. For example, an animal can be treated with compounds capable of modifying the function of a cell involved in a hypersensitive response, compounds that reduce allergic reactions, such as by systemic agents or anti-inflammatory agents (e.g., anti-histamines, anti-steroid reagents, anti-inflammatory reagents and reagents that drive immunoglobulin heavy chain class switching from IgE to IgG). Suitable compounds useful for modifying the function of a cell involved in a hypersensitive response include, but are not limited to, antihistamines, cromolyn sodium, theophylline, cyclosporin A, adrenalin, cortisone, compounds capable of regulating cellular signal transduction, compounds capable of regulating adenosine 3′,5′-cyclic phosphate (cAMP) activity, and compounds that block IgE activity, such as peptides from IgE or IgE specific Fc receptors, antibodies specific for peptides from IgE or IgE-specific Fc receptors, or antibodies capable of blocking binding of IgE to Fc receptors.

Another aspect of the present invention includes a method for prescribing treatment for animals susceptible to or having allergic dermatitis, using a formulation of the present invention. A preferred method for prescribing treatment for flea allergy dermatitis, for example, comprises: (1) intradermally injecting into an animal at one site an effective amount of a formulation containing at least one flea saliva antigen of the present invention, or a mimetope thereof (suitable and preferred formulations are disclosed herein); (2) intradermally injecting into the animal at a second site an effective amount of a control solution; (3) evaluating if the animal has flea allergy dermatitis by measuring and comparing the wheal size resulting from injection of the formulation with the wheal size resulting from injection of the control solution; and (4) prescribing a treatment for the flea allergy dermatitis.

An alternative preferred method for prescribing treatment for flea allergy dermatitis comprises: (1) contacting a first portion of a sample of bodily fluid obtained from an animal to be tested with an effective amount of a formulation containing at least one flea saliva antigen, or a mimetope thereof (suitable and preferred formulations are disclosed herein) to form a first immunocomplex solution; (2) contacting a positive control antibody to form a second immunocomplex solution; (3) evaluating if the animal has flea allergy dermatitis by measuring and comparing the amount of immunocomplex formation in the first and second immunocomplex solutions; and (4) prescribing a treatment for the flea allergy dermatitis. It is to be noted that similar methods can be used to prescribe treatment for allergies caused by other ectoparasites using ectoparasite saliva product formulations as disclosed herein.

Another aspect of the present invention includes a method for monitoring animals susceptible to or having allergic dermatitis, using a formulation of the present invention. In vivo and in vitro tests of the present invention can be used to test animals for allergic dermatitis prior to and following any treatment for allergic dermatitis. A preferred method to monitor treatment of flea allergy dermatitis (which can also be adapted to monitor treatment of other ectoparasite allergies) comprises: (1) intradermally injecting an animal at one site with an effective amount of a formulation containing at least one flea saliva protein, or a mimetope thereof (suitable and preferred formulations are disclosed herein); (2) intradermally injecting an effective amount of a control solution into the animal at a second site; and (3) determining if the animal is desensitized to flea saliva antigens by measuring and comparing the wheal size resulting from injection of the formulation with the wheal size resulting from injection of the control solution.

An alternative preferred method to monitor treatment of flea allergy dermatitis (which can be adapted to monitor treatments of other ectoparasite allergies) comprises: (1) contacting a first portion of a sample of bodily fluid obtained from an animal to be tested with an effective amount of a formulation containing at least one flea saliva protein or mimetope thereof (suitable and preferred formulations are disclosed herein) to form a first immunocomplex solution; (2) contacting a positive control antibody to form a second immunocomplex solution; and (3) determining if the animal is desensitized to flea saliva antigens by measuring and comparing the amount of immunocomplex formation in the first and second immunocomplex solutions.

The present invention also includes antibodies capable of selectively binding to an ectoparasite saliva protein, or mimetope thereof. Such an antibody is herein referred to as an anti-ectoparasite saliva protein antibody. As used herein, the term “selectively binds to” refers to the ability of such an antibody to preferentially bind to ectoparasite saliva proteins and mimetopes thereof. In particular, the present invention includes antibodies capable of selectively binding to flea saliva proteins. Binding can be measured using a variety of methods known to those skilled in the art including immunoblot assays, immunoprecipitation assays, enzyme immunoassays (e.g., ELISA), radioimmunoassays, immunofluorescent antibody assays and immunoelectron microscopy; see, for example, Sambrook et al., ibid.

Antibodies of the present invention can be either polyclonal or monoclonal antibodies. Antibodies of the present invention include functional equivalents such as antibody fragments and genetically-engineered antibodies, including single chain antibodies, that are capable of selectively binding to at least one of the epitopes of the protein or mimetope used to obtain the antibodies. Preferably, an antibody of the present invention has a single site binding affinity of from about 10³ M⁻¹ to about 10¹² M⁻¹ for a flea saliva product of the present invention.

A preferred method to produce antibodies of the present invention includes administering to an animal an effective amount of an ectoparasite saliva protein or mimetope thereof to produce the antibody and recovering the antibodies. Antibodies raised against defined proteins or mimetopes can be advantageous because such antibodies are not substantially contaminated with antibodies against other substances that might otherwise cause interference in a diagnostic assay or side effects if used in a therapeutic composition.

Antibodies of the present invention have a variety of potential uses that are within the scope of the present invention. For example, such antibodies can be used (a) as vaccines to passively immunize an animal in order to protect the animal from allergic dermatitis, (b) as positive controls in test kits, and/or (c) as tools to recover desired ectoparasite saliva proteins from a mixture of proteins and other contaminants.

The following examples are provided for the purposes of illustration and are not intended to limit the scope of the present invention.

### EXAMPLES

It is to be noted that the Examples include a number of molecular biology, microbiology, immunology and biochemistry techniques considered to be known to those skilled in the art. Disclosure of such techniques can be found, for example, in Sambrook et al., ibid., Borovsky, Arch. Insect Biochem. and Phys., 7:187-210, 1988, and related references. Examples 1 through 16, and the SEQ ID NO's cited therein, of related PCT Publication WO 96/11,271, published Apr. 18, 1996, are incorporated herein by this reference in their entirety.

### Example 1

This example describes the amino acid sequence analysis of additional isolated flea saliva proteins from FS-1 extract and eluted from DE-81 filters.

FS-1 flea saliva extract and flea saliva product eluted from DE-81 filters were collected using techniques described in Example 2 of related PCT Publication No. WO 96/11,271. Using standard purification techniques (e.g., C4 reverse phase chromatography; SDS-PAGE gel electrophoresis and blotting; and/or flow through electrophoresis), several proteins were isolated from peak M and partial amino acid sequences were determined as described in Example 4 of related PCT Publication No. WO 96/11,271. Partial N-terminal amino acid sequencing indicated that peak M contained fspJ, fspL and fspN proteins (as described in Example 4 of related PCT Publication No. WO 96/11,271) as well as newly identified proteins referred to herein as fspM(G), fspM(H), fspM(I), fspM(J), fspM(K), fspM(L) and fspM(M). Flea saliva protein fspM(G), having a molecular weight of about 37 kD, had an N-terminal partial amino acid sequence of M R G N H V F L E D G M A D M T G G Q Q M G R D L Y, denoted SEQ ID NO:1. Flea saliva protein fspM(H), having a molecular weight of about 34 kD, had an N-terminal partial amino acid sequence of K Y R N (Y/D) X T N D P Q Y, denoted SEQ ID NO:2. Flea saliva protein fspM(I), having a molecular weight of about 10 kD had an N-terminal partial amino acid sequence of E I K R N D R E P G N L S K I R T V M D K V I K Q T Q, denoted SEQ ID NO:3. Flea saliva protein fspM(J), having a molecular weight of about 25 kD, had an N-terminal partial amino acid sequence of L K D N D I Y (A/H) (A/H) R D I N E I L R V L D P S K, denoted SEQ ID NO:4. Flea saliva protein fspM(K), having a molecular weight of about 30 kD, had an N-terminal partial amino acid sequence of N Y G R V Q I E D Y T X S N H K D X E E K D Q I N G L, denoted SEQ ID NO:5. Flea saliva protein fspM(L), having a molecular weight of about 37 kD, had an N-terminal partial amino acid sequence of K Y R N X Y T N D P Q L K L L D E G, denoted SEQ ID NO:6. Flea saliva protein fspM(M) was recovered from peak M and subjected to amino acid sequence analysis as described in Example 4 of related PCT Publication No. WO 96/11,271. Flea saliva protein fsp(M), having a molecular weight of about 31 kD, had an N-terminal partial amino acid sequence of Y F N D Q I K S V M E P X V F K Y P X A X L, denoted SEQ ID NO:7. A Genbank homology search revealed no significant homology between known amino acid sequences and those determined for fspM(G), fspM(H), fspM(I), fspM(J), fspM(K), fspM(L) and fspM(M).

### Example 2

This example describes the isolation of nucleic acid molecules encoding at least a portion of a fspG flea saliva protein. This example also describes expression of a fspG protein by bacteria.

A. Isolation of fspG4 Nucleic Acid Molecules

The partial N-terminal amino acid sequence of fspG2 (i.e., SEQ ID NO:29 of related PCT Publication No. WO 96/11,271) was used to synthesize degenerate antisense Primer G2-2, having the nucleic acid sequence 5′ TGR TTT CCW ATR AAR TCT TC 3′, denoted SEQ ID NO:8. Primer G2-2 was used in combination with the M13 reverse primer (SEQ ID NO:40; described in Example 7 of related PCT Publication No. Wo 96/11,271), to PCR amplify, using standard techniques, the 5′-terminal portion of the fspG4 gene from a salivary gland cDNA expression library as described above in Example 6A of related PCT Publication No. WO 96/11,271. The resulting PCR product was approximately 225-bp when visualized on a 1% agarose gel. The nucleotide sequence of the 225-bp PCR fragment was obtained, named nfspG4₂₂₅ is presented as SEQ ID NO:9.

The nucleic acid sequence of nfspG4₂₂₅ was used to synthesize sense Primer G5, having nucleic acid sequence 5′ AAT TCG GCA CGA GTG 3′, denoted SEQ ID NO:10. Primer G5 was used in combination with the M13 universal primer (SEQ ID NO:19; described in Example 6 of related PCT Publication No. WO 96/11,271), to PCR amplify, as described above, the 3′-terminal portion of the fspG4 gene from the salivary gland cDNA expression library described above in Example 6A of related PCT Publication No. WO 96/11,271). The resulting PCR product, denoted nfspG4₆₁₀, was approximately 610-bp when visualized on a 1% agarose gel. The nucleotide sequence of the 610-bp PCR fragment was obtained, 565 nucleotides of which are presented as SEQ ID NO:11. The nucleic acid molecule containing nucleic acid sequence SEQ ID NO:11 is referred to herein as nfspG4₅₆₅. Translation of SEQ ID NO:11 suggests that nucleic acid molecule nfspG4₅₆₅ encodes a full-length fspG protein of about 90 amino acids, referred to herein as PfspG4₉₀, assuming an open reading frame having a start codon spanning from about nucleotide 45 through about nucleotide 47 of SEQ ID NO:11 and a stop codon spanning from about nucleotide 315 through about nucleotide 317 of SEQ ID No:11. This open reading frame, excluding the stop codon, comprises nucleic acid molecule nfspG4₂₇₀ of the present invention, the nucleic acid sequence of which is represented herein by SEQ ID NO:13. PfspG4₉₀ is denoted herein as SEQ ID NO:12. Residues 20-42 of SEQ ID NO:12 appear to be identical to SEQ ID NO:29 of related PCT Publication No. WO 96/11,271 (N-terminal partial amino acid sequence of fspG2), except that residue 37 of SEQ ID NO:12 is a glutamic acid rather than a lysine. In addition, residues 38-57 of SEQ ID NO:12 appear to be identical to SEQ ID NO:30 of related PCT Publication No. WO 96/11,271 (N-terminal partial amino acid sequence of fspG3). These similarities support the likelihood of a family of fspG proteins in flea saliva.

Analysis of SEQ ID NO:11 suggests that the sequence includes a leader segment of about 19 amino acids followed by a mature protein. The leader sequence is apparently cleaved to form a mature protein termed PfspG4₇₁, denoted SEQ ID NO:12. PfspG4₇₁ has a calculated molecular weight of 7536 daltons and calculated pI of about 9.0. PfspG4₉₀ has a calculated molecular weight of 9657 daltons and calculated pI of about 9.26. A Genbank homology search revealed no significant homology between SEQ ID NO:11 or SEQ ID NO:12 and known nucleic acid sequences or known amino acid sequences, respectively.

B. Expression

An about 216-bp DNA fragment of nfspG4 was PCR amplified from nucleic acid molecule nfspG4, using: Primer G7, a sense primer having the nucleic acid sequence 5′ AGT GGA TCC GTC AAA AAT GGT CAC TG 3′, denoted as (SEQ ID NO:15 (BamHI site in bold); and Primer G8, an antisense primer having the nucleic acid sequence 51 CCG GAA TTC GGT TAT TCG CAA TAA CAG T 3′ (EcoRI site in bold), denoted SEQ ID NO:16. The PCR product, a fragment of about 216 nucleotides, denoted nfspG4₂₁₆, was digested with BamHI and EcoRI restriction endonucleases, gel purified, and subcloned into expression vector P_{R}/T²ori/S10HIS-RSET-A9 (described in Example 16 of related PCT Publication No. WO 96/11,271) that had been digested with BamHI and EcoRI to produce recombinant molecule pHis-nfspG4₂₁₆.

The recombinant molecule was transformed into E. coli to form recombinant cell E. coli:pHis-nfspG4₂₁₆. The recombinant cell was cultured and induced as described in Example 11A of related PCT Publication No. WO 96/11,271 to produce fusion protein PHIS-fspG4₇₂. The recombinant fusion protein was detected by immunoblot analysis using the T7 Tag monoclonal antibody as described in Example 11A of related PCT Publication No. WO 96/11,271.

### Example 3

This example describes the isolation of nucleic acid sequences encoding at least a portion of flea saliva proteins fspM(A), fspM(B), fspM(C), fspM(D), fspM(E), and fspM(F).

A. nfspM(A)₈₉₇ and nfspM(B)₂₇₀₆

A flea salivary gland cDNA library (prepared as described in Example 6 of related PCT Publication No. WO 96/11,271) was immunoscreened with antiserum collected from a rabbit that was immunized with the proteins in peak M2 of the HPLC separation of flea saliva extract described in Example 3 of related PCT Publication No. WO 96/11,271 (i.e., fspM2 proteins). Immunoscreening was performed as described in Example 12 of related PCT Publication No. WO 96/11,271.

A nucleotide sequence for a nfspM nucleic acid molecule named nfspM(A)₈₉₇ is denoted as SEQ ID NO:17. Translation of SEQ ID NO:17 suggests that nucleic acid molecule nfspM(A)₈₉₇ encodes a full-length fspM protein of about 157 amino acids, referred to herein as PfspM(A)157, assuming an open reading frame having a start codon spanning from about nucleotide 97 through about nucleotide 99 of SEQ ID NO:17 and a stop codon spanning from about nucleotide 568 through about nucleotide 570 of SEQ ID NO:17. This open reading frame, excluding the stop codon, comprises nucleic acid molecule nfspM(A)₄₇₁ of the present invention, the nucleic acid sequence of which is represented herein by SEQ ID NO:19. The amino acid sequence of PfspM(A)157 is denoted SEQ ID NO:18. PfspM(A)₁₅₇ has a calculated molecular weight of about 18,291.68 daltons and calculated pI of about 10.3. A Genbank homology search revealed no significant homology between SEQ ID NO:17 or SEQ ID NO:18 and known nucleic acid or amino acid sequences, respectively.

A nucleotide sequence for another nfspM nucleic acid molecule named nfspM(B)₂₇₀₆ is denoted as SEQ ID NO:20. Translation of SEQ ID NO:20 suggests that nucleic acid molecule nfspM(B)₂₇₀₆ encodes a non-full-length fspM protein of about 900 amino acids, referred to herein as PfspM(B)₉₀₀, assuming an open reading frame having a start codon spanning from about nucleotide 5 through about nucleotide 7 of SEQ ID NO:20. The amino acid sequence of PfspM(B)₉₀₀ is denoted SEQ ID NO:21. PfspM(B)₉₀₀ has a calculated molecular weight of about 104,647 daltons and calculated pI of about 5.8.

The nucleic acid and amino acid sequences of the nfspM(B)₂₇₀₆ nucleic acid molecule and PfspM(B)₉₀₀ protein, respectively, were compared to known nucleic acid and amino acid sequences using a Genbank homology search. SEQ ID NO:21 was found to be similar to the amino acid sequence of RhoA-binding alpha kinase (ROK). The most highly conserved region of continuous similarity between SEQ ID NO:21 and ROK amino acid sequences spans from about amino acid 32 through about amino acid 351 of SEQ ID NO:21 and from about amino acid 1 through about amino acid 900 of the ROK, there being about 75% identity between the two regions. Comparison of the nucleic acid sequence encoding amino acids from about 326 through about 1285 of the ROK kinase with the corresponding regions, spanning nucleotides from about 98 through about 1075 of nfspM(B)₂₇₀₆ indicate that those regions are about 71% identical.

B. nfspM(C)₄₁₄ and nfspM(D)₂₇₃

A flea salivary gland cDNA library (prepared as described in Example 6 of related PCT Publication No. WO 96/11,271) was immunoscreened with antiserum collected from a rabbit that was immunized with the proteins in peak Ml of the HPLC separation of flea saliva extract described in Example 3 of related PCT Publication No. WO 96/11,271 (i.e., fspM1 proteins). Immunoscreening was performed as described in Example 12 of related PCT Publication No. WO 96/11,271.

Nucleotide sequence for a nfspm nucleic acid molecule named nfspM(C)₄₁₄ is denoted as SEQ ID NO:22. Translation of SEQ ID NO:22 suggests that nucleic acid molecule nfspM(C)414 encodes a non-full-length fspM protein of about 137 amino acids, referred to herein as PfspM(C)₁₃₇, assuming the first residue spans from about nucleotide 2 through about nucleotide 4 of SEQ ID NO:22. The amino acid sequence of PfspM(C)₁₃₇ is denoted SEQ ID NO:23. PfspM(C)₁₃₇ has a calculated molecular weight of about 14,452 daltons and calculated pI of about 2.81. A Genbank homology search revealed no significant homology between SEQ ID NO:22 or SEQ ID NO:23 and known nucleic acid sequences or known amino acid sequences, respectively.

A nucleotide sequence for another nfspM nucleic acid molecule named nfspM(D)₂₇₃ is denoted as SEQ ID NO:24. Translation of SEQ ID NO:24 suggests that nucleic acid molecule nfspM(D)₂₇₃ encodes a non-full-length fspm protein of about 90 amino acids, referred to herein as PfspM(D)₉₀, assuming the first residue spans from about nucleotide 3 through about nucleotide 5 of SEQ ID NO:24. The amino acid sequence of PfspM(D)₉₀ is denoted SEQ ID NO:25. PfspM(D)₉₀ has a calculated molecular weight of about 9,503 daltons and calculated pI of about 3.01. SEQ ID NO:24 and SEQ ID NO:25 appear to be substantially similar to SEQ ID NO:22 and SEQ ID NO:23, respectively, suggesting a family of fspM proteins in flea saliva.

C. nfspM(E)₁₇₀₄ and nfspM(F)1758

A flea salivary gland cDNA library (prepared as described in Example 6 as described of related PCT Publication No. WO 96/11,271) was immunoscreened with antiserum collected from a rabbit that was immunized with the proteins in peak M2 of the HPLC separation of flea saliva extract described in Example 3 of related PCT Publication No. WO 96/11,271 (i.e., fspM2 proteins). Immunoscreening was performed as described in Example 12 of related PCT Publication No. WO 96/11,271.

A nucleotide sequence for another nfspM nucleic acid molecule named nfspM(E)₁₇₀₄ is denoted as SEQ ID NO:26. Translation of SEQ ID NO:26 suggests that nucleic acid molecule nfspM(E)₁₇₀₄ encodes a full-length fspM protein of about 461 amino acids, referred to herein as PfspM(E)₄₆₁, assuming the first residue spans from about nucleotide 24 through about nucleotide 26 of SEQ ID NO:26 and a stop codon spanning from about nucleotide 1407 through about nucleotide 1409 of SEQ ID NO:26. This open reading frame, excluding the stop codon, comprises nucleic acid molecule nfspM(E)₁₃₈₃ of the present invention, the nucleic acid sequence of which is represented herein by SEQ ID NO:28. The amino acid sequence of PfspM(E)₄₆₁ is denoted SEQ ID NO:27. PfspM(E)₄₆₁ has a calculated molecular weight of about 54,139 daltons and calculated pI of about 7.00. A Genbank homology search revealed no significant homology between SEQ ID NO:26 or SEQ ID NO:27 and known nucleic acid sequences or known amino acid sequences, respectively.

A nucleotide sequence for another nfspM nucleic acid molecule named nfspM(F)₁₇₅₈ is denoted as SEQ ID NO:29. Translation of SEQ ID NO:29 suggests that nucleic acid molecule nfspM(F)₁₇₅₈ encodes a non-full-length fspM protein of about 5S86 amino acids, referred to herein as PfspM(F)₅₈₆, assuming an open reading frame having a start codon spanning from about nucleotide 1 through about nucleotide 3 of SEQ ID NO:29. The amino acid sequence of PfspM(F)₅₈₆ is denoted SEQ ID NO:30. PfspM(F)₅₈₆has a calculated molecular weight of about 66,547 daltons and calculated pI of about 4.80. A Genbank homology search revealed no significant homology-between SEQ ID NO:29 or SEQ ID NO:30 and known nucleic acid sequences or known amino acid sequences, respectively.

### Example 4

This Example demonstrates the expression of a fspM protein in E. Coli cells.

Flea saliva protein PHIS-PfspM(D)₉₀ fusion protein was produced in the following manner. An about 305-bp DNA fragment, referred to herein as nfspM(D)₃₀₅, was isolated from nfspM(D)₂₉₃ (denoted SEQ ID NO:31) subcloned into pBluescript plasmid by digesting the nfspM(D)-containing plasmid with BamH1 and XhoI restriction endonucleases. The digestion product was gel purified and subcloned into expression vector pTrcHisB that had been digested with BamH1 and XhoI, and dephosphorylated. The resultant recombinant molecule, referred to herein as pHis-nfspM(D)₃₀₅, was transformed into E. coli HB101 competent cells (available from Gibco BRL, Gaithersburg, Md.) to form recombinant cell E. coli:pHis-nfspM(D)₃₀₅. The recombinant cell was cultured and expression of nfspM₃₀₅ induced using conditions described in Example 11A of related PCT Publication No. WO 96/11,271. Immunoblot analysis of recombinant cell E. coli:pHis-nfspM(D)₃₀₅ lysates using a T7 tag monoclonal antibody (Novagen, Inc) directed against the fusion portion of the recombinant PHis-nfspM(D)₃₀₅ fusion protein identified a protein of the appropriate size, namely an about 15,851 kD protein.

### Example 5

This example describes the isolation of nucleic acid sequences encoding at least a portion of flea saliva proteins fspN(C), fspN(D), fspN(E), fspN(F), fspN(G), fspN(H), fspN(I), fspN(J), fspN(K), fspN(L), fspN(M), fspN(N) and fspN(O).

A. Preparation of IgE Enriched Antiserum

Serum was obtained from the artificially sensitized dog CQQ2 (described in Example 8 of related PCT Publication No. WO 96/11,271). About 10 ml of antiserum was incubated with protein G-Sepharose (5 ml) over night at 4° C.

B. Immunoscreening with IgE Enriched Antiserum

About 2.4 ml of Escherichia coli (XL1 Blue, O.D.₆₀₀=0.5) was incubated with 6.48×10⁵ pfu of phage from a flea salivary gland ZAP-cDNA library (1.8×10⁷ pfu/ml), at 37° C. for 15 min and plated in 12 Luria-Bertani (LB) medium agar plates (150 mm). The plates were incubated at 37° C. over night. Each plate was then overlaid with an IPTG (10 (M) treated nitrocellulose filters for about 4 hours at 37° C. The filters were then removed and washed with TBST (20 mM Tris-HCl pH 7.5, 150 mM NaCl, 0.05% Tween-20). The filters were blocked with 5% dry milk in TBST for 2 hours at room temperature. Different filters were then incubated first with either IgE enriched CQQ2 antiserum or antiserum obtained from dogs infected with Dirofilaria immitis) at 4° C., overnight, then with a monoclonal anti-canine IgE antibody (D-9; gift from the laboratory of Dr. D. J. DeBoer, School of Veterinary Medicine, University of Wisconsin, Madison, Wis.), and then with a donkey anti-mouse IgG antibody conjugated to horseradish peroxidase (available from Jackson ImmunoResearch, West Grove, PN) for 2 hours at room temperature at each step. All of the filters were washed with TBST (3×15 min/wash) between each incubation. All of the filters were then treated to a final wash in TBS. Immunocomplexed plaques were identified by immersing the filters into the developing solution (TMB Peroxidase Substrate/TMB Peroxidase Solution/TMB Membrane Enhancer from Kirkegaard & Perry Laboratories) at 1/1/0.1 volume ratio to produce a color reaction. Eighteen plaques were identified and further plaque purified under the same immunoscreening condition as described above.

C. nfspN(C)₃₃₅, nfspN(D)₃₉₆, nfspN(E)₂₈₅, nfspN(F)₂₂₈, nfspN(G)₃₃₉, nfspN(G)₄₉₃,

Single plaque of purified clones were isolated and stored in SM phage buffer (50 mM Tris, pH 7.4, 0.58% NaCl, 0.2% MgCl₂.7H₂O and 0.01% Gelatin). The in vivo excision of the pBluescript phagemid from each positive clone was prepared by using ExAssis™/SOLR™ system (Stratagene). The pBluescript plasmid was purified by plasmid midi kit (Qiagen), and denatured with NaOH (0.4 N) at 37° C. for 15 min. The denatured plasmid was precipitated by ethanol and nucleic acid sequence obtained.

A nucleotide sequence for a nfspN nucleic acid molecule named nfspN(C)₃₃₅ is denoted as SEQ ID NO:32. A Genbank homology search revealed some similarity between SEQ ID NO:32 and ribosomal protein S6.

A nucleotide sequence for another nfspN nucleic acid molecule named nfspN(D)₃₉₆ is denoted as SEQ ID NO:33. A Genbank homology search revealed some similarity between SEQ ID NO:33 and erythropoietin.

A nucleotide sequence for another nfspN nucleic acid molecule named nfspN(E)₂₈₅ is denoted as SEQ ID NO:34. A Genbank homology search revealed some similarity between SEQ ID NO:34 and glutamic acid-rich protein or heat-shock protein, HSP81.

A nucleotide sequence for another nfspN nucleic acid molecule named nfspN(F)₂₂₈ is denoted as SEQ ID NO:35.

Nucleic acid sequence for portions of another nfspN nucleic acid molecule, denoted herein as nfspN(G), were obtained. The nucleic acid molecule representing a 5′ portion of nfspN(G) named nfspN(G)₃₃₉ is denoted as SEQ ID NO:36. Translation of SEQ ID NO:36 suggests that nucleic acid molecule nfspN(G)₃₃₉ encodes a non-full-length fspN(G) protein of about 113 amino acids, referred to herein as PfspN(G)₁₁₃, assuming the first residue spans from about nucleotide 1 through about nucleotide 3 of SEQ ID NO:36. The amino acid sequence of PfspN(G)₁₁₃ is denoted SEQ ID NO:37.

The nucleic acid molecule representing a 3′ portion of nfspN(G) named nfspN(G)₄₉₃ is denoted as SEQ ID NO:38. Translation of SEQ ID NO:38 suggests that nucleic acid molecule nfspN(G)₄₉₃ encodes a non-full-length fspN(G) protein of about 130 amino acids, referred to herein as PfspN(G)₁₃₀, assuming the first residue spans from about nucleotide 1 through about nucleotide 3 of SEQ ID NO:38 and a stop codon spanning from about nucleotide 391 through about nucleotide 393 of SEQ ID NO:38. The amino acid sequence of PfspN(G)₁₃₀ is denoted SEQ ID NO:39. A Genbank homology search revealed some similarity between SEQ ID NO:36 and SEQ ID NO:38 and vitellogenin.

A nucleotide sequence for another nfspN nucleic acid molecule named nfspN(H)₃₀₆ is denoted as SEQ ID NO:40.

A nucleotide sequence for another nfspN nucleic acid molecule named nfspN(I)₄₉₀ is denoted as SEQ ID NO:41.

A nucleotide sequence for another nfspN nucleic acid molecule named nfspN(J)₆₁₆ is denoted as SEQ ID NO:42.

A nucleotide sequence for another nfspN nucleic acid molecule named nfspN(K)₄₇₅ is denoted as SEQ ID NO:43.

A nucleotide sequence for another nfspN nucleic acid molecule named nfspN(L)₂₉₅ is denoted as SEQ ID NO:44.

A nucleotide sequence for another nfspN nucleic acid molecule named nfspN(M)₃₇₂ is denoted as SEQ ID NO:45.

Nucleic acid sequence for portions of another nfspN nucleic acid molecule, denoted herein as nfspN(N), were obtained. The nucleic acid molecule representing a 5′ portion of nfspN(N) named nfspN(N)₂₅₂ is denoted as SEQ ID NO:46. The nucleic acid molecule representing a 3′ portion of nfspN(N) named nfspN(N)₆₁₃ is denoted as SEQ ID NO:47.

Nucleic acid sequence for portions of another nfspN nucleic acid molecule, denoted herein as nfspN(O), were obtained. The nucleic acid molecule representing a 5′ portion of nfspN(O) named nfspN(O)₅₃₈ is denoted as SEQ ID NO:48. Translation of SEQ ID NO:48 suggests that nucleic acid molecule nfspN(O)₅₃₈ encodes a non-full-length fspN(O) protein of about 178 amino acids, referred to herein as PfspN(O)₁₇₁, assuming the first residue spans from about nucleotide 1 through about nucleotide 3 of SEQ ID NO:48. The amino acid sequence of PfspN(N)₁₇₈ is denoted SEQ ID NO:49.

The nucleic acid molecule representing a 3′ portion of nfspN(O) named nfspN(O)₄₃₂ is denoted as SEQ ID NO:50. Translation of SEQ ID NO:50 suggests that nucleic acid molecule nfspN(O)₄₃₂ encodes a non-full-length fspN(O) protein of about 129 amino acids, referred to herein as PfspN(O)₁₂₉, assuming the first residue spans from about nucleotide 1 through about nucleotide 3 of SEQ ID NO:50 and a stop codon spanning from about nucleotide 388 through about nucleotide 390 of SEQ ID NO:50. The amino acid sequence of PfspN(O)₁₂₉ is denoted SEQ ID NO:51.

### Example 6

This example describes studies confirming the specificity of IgE enriched antiserum from CQQ2 to fspN protein.

Three different petri dishes (100 mm) were overlaid with 300 microliter per plate of E. coli (XL1 Blue, O.D. ₆₀₀=500) A drop (about 100 pfu/drop) of each of the eighteen isolated phage clones was dropped onto each plate (18 phage clones/plate). Using the methods described in Example 5 above, the plates were incubated, filter lifted and the filters immunoscreened with IgE enriched antiserum from CQQ2, antiserum from a D. Immitis infected dog and antiserum from rabbits injected with flea saliva product from peak N (as described in Example 3 of related PCT Publication No. WO 96/11,271).

The results of the experiment indicate that both the IgE enriched CQQ2 antiserum and the antiserum specific for peak N flea saliva product bind to the products of the purified phage clones significantly better than the antiserum from a D. Immitis infected dog.

### Example 7

This example describes the isolation of nucleic acid molecules encoding a fspG flea saliva protein. This example also describes expression of a fspG protein by bacteria.

A DNA probe labeled with ³²P comprising nucleotides from nfspG4₆₁₀ (described in Example 2) was used to screen a flea salivary gland cDNA library (described in Example 6 of related PCT Publication No. WO 96/11,706) using standard hybridization techniques. A clone was isolated having about a 595 nucleotide insert, referred to herein as nfspG5₅₉₅ having a nucleic acid sequence of the coding strand which is denoted herein as SEQ ID NO:52. Translation of SEQ ID NO:52 suggests that nucleic acid molecule nfspG5₅₉₅, encodes a full-length flea salivary protein of about 90 amino acids, referred to herein as PfspG5₉₀, having amino acid sequence SEQ ID NO:53, assuming an open reading frame in which the initiation codon spans from about nucleotide 46 through about nucleotide 48 of SEQ ID NO:52 and the termination codon spans from about nucleotide 316 through about nucleotide 318 of SEQ ID NO:52. The complement of SEQ ID NO:52 is represented herein by SEQ ID NO:54. The coding region encoding PfspG5₉₀, is represented by nucleic acid molecule nfspG5₂₇₀, having a coding strand with the nucleic acid sequence represented by SEQ ID NO:55 and a complementary strand with nucleic acid sequence SEQ ID NO:57. The amino acid sequence of PfspG5₉₀ (i.e., SEQ ID NO:53) predicts that PfspG5₉₀ has an estimated molecular weight of about 9.6 kD and an estimated pI of about 9.28.

Analysis of SEQ ID NO:53 suggests the presence of a signal peptide encoded by a stretch of amino acids spanning from about amino acid 1 through about amino acid 19. The proposed mature protein, denoted herein as PfsG5₇₁, contains about 71 amino acids which is represented herein as SEQ ID NO:59. The complement of SEQ ID NO:58 is represented by SEQ ID NO:60. The amino acid sequence of PfspG5₇₁ (i.e., SEQ ID NO:59) predicts that PfspG5₇₁ has an estimated molecular weight of about 7.48 kD, and an estimated pI of about 8.28.

Comparison of amino acid sequence SEQ ID NO:53 with amino acid sequences reported in GenBank indicates that SEQ ID NO:53 showed the most homology, i.e., about 38% identity between SEQ ID NO:53 and a Ctenocephalides felis flea salivary protein FS-H precursor (GenBank accession U63544). Comparison of nucleic acid sequence SEQ ID NO:52 with nucleic acid sequences reported in GenBank indicates that SEQ ID NO:52 showed the most homology, i.e., about 63% identity between SEQ ID NO:52 and a Ctenocephalides felis flea salivary protein ES-H precursor gene (GenBank accession U63544).

Flea salivary protein PfspG5₇₁ was produced in the following manner. An about 213 bp nucleic acid molecule, referred to herein as nfspG5₂₁₃ (designed to encode an apparently mature flea salivary protein) was PCR amplified from nfspG5₅₉₅ using sense primer G7 having the nucleotide sequence 5′ A GTG GAT CCG TCA AAA ATG GTC ACT G-3′ (containing an BamHI-site shown in bold; denoted SEQ ID NO:79) and anti-sense primer G8 having the nucleotide sequence 5′ CC GGA ATT CGG TTA TTC GCA ATA ACA GT-3′ (containing a EcoRI shown in bold; denoted SEQ ID NO:80). The resulting PCR product nfspG5₂₁₃ was digested with BamHI and EcoRI restriction endonucleases, gel purified, and subcloned into expression vector lambdaP_{R}/T²ori/S10HIS-RSET-A9, that had been digested with BamHI and EcoRI and dephosphorylated. The resultant recombinant molecule, referred to herein as pCro-nfspG5₂₁₃, was transformed into E. coli BL-21 competent cells (available from Novagen, Madison, Wis.) to form recombinant cell E. coli:pCro-nfspG5₂₁₃. The recombinant cell was cultured and induced as described in Example 11A of related PCT Publication No. WO 96/11,271. Immunoblot analysis of the proteins using a T7 antibody showed expression of an about 12 kD protein in the induced sample but not in the uninduced sample.

### Example 8

This example describes the further sequencing of a nucleic acid sequence encoding a fspI flea saliva protein. This example also describes expression of a fspI protein by bacteria.

The nucleic acid molecule denoted nfspI₅₇₃ described in Example 6 of related PCT Publication No. WO 96/11,706 was further sequenced using standard nucleotide sequencing methods. A nucleic acid molecule was identified of about 1007 nucleotides, referred to herein as nfspI₁₀₀₇, the coding strand is denoted herein as SEQ ID NO:61. Translation of SEQ ID NO:61 suggests that SEQ ID NO:61 encodes a non-full-length flea salivary protein of about 155 amino acids, referred to herein as PfspI₁₅₅, having amino acid sequence SEQ ID NO:62, assuming the first codon spans from about nucleotide 1 through about nucleotide 3 of SEQ ID NO:61 and the termination codon spans from about nucleotide 466 through about nucleotide 468 of SEQ ID NO:61. The complement of SEQ ID NO:61 is represented herein by SEQ ID NO:63.

Flea salivary protein PfspI₁₅₈ was produced in the following manner. An about 474-bp nucleic acid molecule, referred to herein as nfspI₄₇₄ (designed to encode an apparently mature flea salivary protein) was PCR amplified from nfspI1007 using sense primer I1 having the nucleotide sequence 5′ GCG CGG ATC CGC ATA TGG AAG ACA TCT GGA AAG TTA ATA AAA AAT GTA CAT CAG-3′ (containing an BamHI-site shown in bold as well as nucleic acid sequence encoding three amino acids, Glu-Asp-Isoleucine, shown in italics; denoted SEQ ID NO:81) and anti-sense primer 12 having the nucleotide sequence 5′ CCG GAA TTC TTA TTT ATT TTT TGG TCG ACA ATA ACA AAA GTT TCC-3′ (containing a EcoRI shown in bold; denoted SEQ ID NO:82). The resulting PCR product nfspI₄₇₄, which contained the nucleic acid sequences incorporated into primer I1 that encode three amino acids, was digested with BamHI and EcoRI restriction endonucleases, gel purified, and subcloned into expression vector lambdaP_{R}/T²ori/S10HIS-RSET-A9, that had been digested with BamHI and XbaI and dephosphorylated. The resultant recombinant molecule, referred to herein as pCro-nfspI₄₇₄, was transformed into E. coli BL-21 competent cells (available from Novagen, Madison, Wis.) to form recombinant cell E. coli:pCro-nfspI₄₇₄. The recombinant cell was cultured and protein production resolved using the methods described in Example 11A of related PCT Publication No. WO 96/11,271. Immunoblot analysis of the proteins using a T7 antibody showed expression of an about 30 kD protein in the induced sample but not in the uninduced sample.

### Example 9

This example describes the isolation of nucleic acid molecules encoding a fspN flea saliva protein.

A DNA probe comprising nucleotides from nfspN(B)₆₁₂ (SEQ ID NO:52 of related PCT Publication No. WO 96/11,706) was labeled with ³²P and used to screen the flea salivary gland cDNA library using standard hybridization techniques. A clone was isolated having about a 1205 nucleotide insert, referred to herein as nfspN5₁₂₀₅ having a nucleic acid sequence of the coding strand which is denoted herein as SEQ ID NO:64. Translation of SEQ ID NO:64 suggests that nucleic acid molecule nfspN5₁₂₀₅ encodes a non-full-length flea salivary protein of about 353 amino acids, referred to herein as PfspN5₃₅₃, having amino acid sequence SEQ ID NO:65, assuming an open reading frame in which the initiation codon spans from about nucleotide 4 through about nucleotide 6 of SEQ ID NO:64 and the termination codon spans from about nucleotide 1060 through about nucleotide 1062 of SEQ ID NO:64. The complement of SEQ ID NO:64 is represented herein by SEQ ID NO:66. The coding region encoding PfspN5₃₅₃, is represented by nucleic acid molecule nfspN5₁₀₅₉, having a coding strand with the nucleic acid sequence represented by SEQ ID NO:67 and a complementary strand with nucleic acid sequence SEQ ID NO:69. The amino acid sequence of PfspN5₃₅₃ (i.e., SEQ ID NO:65) predicts that PfspN5₃₅₃ has an estimated molecular weight of about 39.7 kD and an estimated pI of about 9.45.

Comparison of amino acid sequence SEQ ID NO:65 with amino acid sequences reported in GenBank indicates that SEQ ID NO:65 showed the most homology, i.e., about 32% identity between SEQ ID NO:65 and a Human prostatic acid phosphatase precursor protein (GenBank accession P15309). A GenBank homology search revealed no significant homology between SEQ ID NO:64 and known nucleic acid sequences.

### Example 10

This example describes the isolation of nucleic acid molecules encoding a fspN flea saliva protein identified using IgE antibodies isolated from a dog having clinical flea allergy dermatitis.

A pool of sera (referred to herein as Pool #4) was collected from numerous known to have clinic flea allergy dermatitis (FAD). Pool #4 sera was used to identify flea saliva antigens that bind specifically to IgE antibodies in the FAD dog sera as follows. Flea saliva extract was collected using the general methods described in Examples 1 and 2 of related PCT Publication No. WO 96/11,706, except a carboxymethyl cation exchange (CM) membrane (available from Schleicher and Scheull, Keene, N.H.) was used rather than a Durapore® membrane. In addition, flea saliva extract was eluted from the membrane by contacting the membrane in an extraction buffer of 2.5 M NaCl, 5% isopropyl alcohol (IPA) and 20 mM Tris, pH 8.0. The membrane was eluted overnight at room temperature. The flea saliva extract was resolved by high pressure liquid chromatography (HPLC) using the method generally described in Example 2 of related PCT Publication No. WO 96/11,706. Proteins contained in the HPLC fractions were resolved on a 16% Tris-glycine SDS PAGE gel. Proteins on the gel were then blotted to an Immobilon P™ filter (available from Millipore Co., Bedford, Mass.) using standard Western Blot techniques. IgE antibodies bound to protein on the blot was then detected as follows. The blot was first incubated with about a 1:200 dilution of Pool #4 sera using standard antibody hybridization techniques, washed, and then incubated with about a 1:500 dilution of a 145 μg/milliliter solution of biotinylated human Fc R alpha chain protein using standard Western Blot techniques. Following washing, the blot was incubated with about a 1:5,000 dilution of streptavidin conjugated to alkaline phosphatase (available from Sigma, St. Louis, Mo.). About 10 milliliter of BCIP/NBT substrate (available from Gibco BRL, Gaithersburg, Md.) was then added to the blot, incubated until visible bands appeared, at room temperature, and then the blot was rinsed in water to stop the reaction. Protein bands were detected in samples containing Fractions 34, 37, 38, 47, 49, 51, 52 and 53.

Amino (N-) terminal amino acid sequencing analysis was performed on protein contained in the about 40 kD protein band identified in the sample containing Fraction 52, using standard procedures known to those in the art (see, for example, Geisow et al., 1989, in Protein Sequencing: A Practical Approach, JBC Findlay and MJ Geisow (eds.), IRL Press, Oxford, England, pp. 85-98; Hewick et al., 1981, J. Biol. Chem., Vol. 256, pp. 7990-7997). The N-terminal partial amino acid sequence of the protein was determined to be X Glu Leu Lys Phe Val Phe Val Met Val Lys Gly Pro Asp His Glu Ala Cys Asn Tyr Ala Gly Gly X Gln (denoted herein as SEQ ID NO:70; wherein “X” represents any amino acid residue)

Synthetic oligonucleotide primers were designed using SEQ ID NO:70 and used to isolate a nucleic acid molecule encoding SEQ ID NO:70 as follows. Sense primer 1 having the nucleotide sequence 5′ AAA TTT GTA(T) TTT GTA(T) ATG GTA(T) AAA GGA(T) CCA(T) GAT CAT GAA GC-3′ (denoted SEQ ID NO:83) was used in combination with the M13 forward universal standard primer 5′ GTAAAACGACGGCCAGT 3′ (denoted SEQ ID NO:84) to produce a PCR product from the a flea salivary gland cDNA library described above in Example 9. PCR amplification was conducted using standard techniques. The resulting PCR amplification product was a fragment of about 406 nucleotides, denoted herein as nfspN6₄₀₆. The PCR product was cloned into the InVitrogen, Corp., TA™ cloning vector (procedures provided by InVitrogen, Corp.) and subjected to DNA sequence analysis using standard techniques.

The nucleic acid sequence of the coding strand of nfspN6₄₀₆ is denoted herein as SEQ ID NO:71. Translation of SEQ ID NO:71 suggests that nucleic acid molecule nfspN6₄₀₆ encodes a non-full-length flea salivary protein of about 135 amino acids, referred to herein as PfspN6₁₃₅, having amino acid sequence SEQ ID NO:72, assuming the first codon spans from about nucleotide 1 through about nucleotide 3 of SEQ ID NO:71 and the last codon spans from about nucleotide 403 through about nucleotide 405 of SEQ ID NO:71. The complement of SEQ ID NO:71 is represented herein by SEQ ID NO:73.

A GenBank homology search revealed no significant homology between amino acid sequence SEQ ID NO:72 and nucleic acid sequence SEQ ID NO:71 and known amino acid sequences or nucleic acid sequences, respectively.

### Example 11

This example describes the isolation of nucleic acid molecules encoding a fspJ flea saliva protein.

Degenerate oligonucleotide primers were designed from the amino acid sequence deduced for fspJ (described in Example 4 of related PCT Publication No.WO 96/11,706) and were used to isolate a fspJ nucleic acid molecule as follows. Two synthetic oligonucleotides were synthesized that corresponded to the region of fspJ spanning from about residues 7 through about 26 of SEQ ID NO:8 of related PCT Publication No.WO 96/11,706. Primer 1, a “sense” primer corresponding to amino acid residues fro about residue 7 to about 16 of SEQ ID NO:8 of related PCT Publication No.WO 96/11,706, has the nucleotide sequence 5′ CAT GAA CCA(T) GGA(T) AAT ACA(T) CGA(T) AAA(G) ATA(C/T) A(C)G 3′ (denoted herein as SEQ ID NO:84). Primer 2, a “sense” primer corresponding to amino acid residues form about residue 17 through about 26 of SEQ ID NO:8 of related PCT Publication No. WO 96/11,706, has the nucleic acid sequence 5′ GAA GTA(T) ATG GAC(T) AAA TTA(G) AGA(G) CAA(G) GC-3′ (denoted herein as SEQ ID NO:86).

PCR amplification of fragments from the flea salivary gland cDNA library described above in Example 9 was conducted using standard techniques. PCR amplification products were generated using a combination of Primer 1 and M13 primer (denoted SEQ ID NO:85). The resultant PCR products were used for a nested PCR amplification using Primer 2 and the T7 standard primer 5′ GTA ATA CGA CTC ACT ATA TAG GGC 3′ (denoted SEQ ID NO:88). The resultant PCR product, a fragment of about 420 nucleotides, denoted herein as nfspJ₄₂₀. The PCR product was cloned into the InVitrogen, Corp., TA™ cloning vector (procedures provided by InVitrogen, Corp.) and subjected to DNA sequence analysis using standard techniques.

The nucleic acid sequence of the coding strand of nfspJ₄₂₀ is denoted herein as SEQ ID NO:74. Translation of SEQ ID NO:74 suggests that nucleic acid molecule nfspJ₄₂₀ encodes a non-full-length flea salivary protein of about 72 amino acids, referred to herein as PfspJ₇₂, having amino acid sequence SEQ ID NO:75, assuming the first codon spans from about nucleotide 1 through about nucleotide 3 of SEQ ID NO:74 and the last codon spans from about nucleotide 214 through about nucleotide 216 of SEQ ID NO:74. The complement of SEQ ID NO:74 is represented herein by SEQ ID NO:76.

A GenBank homology search revealed no significant homology between amino acid sequence SEQ ID NO:75 and nucleic acid sequence SEQ ID NO:74 and known amino acid sequences or nucleic acid sequences, respectively.

### Example 12

This example describes the amino acid sequence analysis of an isolated and HPLC purified fspN7 flea saliva protein.

Fractions of flea saliva proteins described above in Example 10 were tested for the ability to stimulate T cell clones that respond specifically to the flea saliva extract described in Example 10 (FS-specific T cells). T cell activation were performed using standard methods such as those described in Current Protocols in Immunology, Vol. 1, Chapter 3 [3.13.2], ed. J. E. Coligan et al., pub. Wiley Interscience, 1993. Briefly, about 10⁴ FS-1-specific T cells (clone CP02-7; isolated from dog CPO2 described in Example 8 of related PCT Patent Publication No. WO 96/11,271) were added to individual wells of a 96 well tissue culture plate, in the presence of about 2×104 autologous antigen presenting cells (isolated by ficoll gradient from dog CPO2) and about 100 units/milliliter of recombinant human interleukin-2 (Proleukin®; available from Chiron Inc., Emeryville, Calif.). About 1 microliter of each fraction of protein resolved by HPLC was to added to each well in triplicate. The cells were incubated for about 4 to about 6 days. About 16 hours prior to harvesting, about 1 uCi of tritiated thymidine (available from Amersham Inc., Arlington Heights, Ill.) was added to each well. The cells were then harvested and the amount of tritium incorporated into the cellular protein was determined. The results indicated that protein contained in a HPLC fraction containing fspN protein (Fraction 51) stimulated the FS-specific T cells.

Amino (N-) terminal amino acid sequencing analysis was performed on protein contained in Fraction 51 using standard procedures known to those in the art (see, for example, Geisow et al., ibid.; Hewick et al., 1981, ibid.). The N-terminal partial amino acid sequence of the band was determined to be Asn Asp Lys Leu Gln Phe Val Phe Val Met Ala Arg Gly Pro Asp His Glu Ala Cys Asn Tyr Pro Gly Gly Pro (denoted herein as SEQ ID NO:78).

### Example 13

This example describes the amino acid sequence analysis of an isolated and HPLC purified fspM2 flea saliva protein.

Proteins contained within Fraction 47 described above in Example 10 were resolved on a 16% Tris-glycine SDS PAGE gel. A major band at about 34 kD was identified. Amino (N-) terminal amino acid sequencing analysis was performed on protein contained in the about 34 kD using standard procedures known to those in the art (see, for example, Geisow et al., ibid.; Hewick et al., 1981, ibid.). The N-terminal partial amino acid sequence of the band was determined to be Tyr Phe Asn Lys leu Val Gln Ser Trp Thr Glu Pro Met Val Phe Lys Tyr Pro Tyr (denoted herein as SEQ ID NO:87).

What is claimed is:

1. An isolated nucleic acid molecule that hybridizes under stringent conditions with a gene selected from the group consisting of a flea saliva gene comprising a nucleic acid sequence selected from the group consisting of SEQ ID NO:52, SEQ ID NO:54, SEQ ID NO:55, SEQ ID NO:57, SEQ ID NO:58, SEQ ID NO:60, SEQ ID NO:61, SEQ ID NO:63, SEQ ID NO:64, SEQ ID NO:66, SEQ ID NO:67, SEQ ID NO:69, SEQ ID NO:71, SEQ ID NO:73, SEQ ID NO:74, SEQ ID NO:76 and a nucleic acid sequence encoding an amino acid sequence selected from the group consisting of SEQ ID NO:78 and SEQ ID NO:87.

2. An isolated nucleic acid molecule that hybridizes under stringent hybridization conditions with a nucleic acid molecule having a nucleic acid sequence encoding a protein comprising an amino acid sequence selected from the group consisting of SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87.

3. An isolated protein encoded by a nucleic acid molecule that hybridizes under stringent hybridization conditions with a nucleic acid molecule having a nucleic acid sequence encoding a protein comprising an amino acid sequence selected from the group consisting of SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87.

4. A therapeutic composition for treating allergic dermatitis comprising a formulation comprising at least one isolated ectoparasite saliva protein, wherein said ectoparasite saliva protein comprises at least a portion of an amino acid sequence, wherein said portion is encoded by a nucleic acid molecule that hybridizes under stringent hybridization conditions with a nucleic acid molecule having a nucleic acid sequence selected from the group consisting of SEQ ID NO:52, SEQ ID NO:54, SEQ ID NO:55, SEQ ID NO:57, SEQ ID NO:58, SEQ ID NO:60, SEQ ID NO:61, SEQ ID NO:63, SEQ ID NO:64, SEQ ID NO:66, SEQ ID NO:67, SEQ ID NO:69, SEQ ID NO:71, SEQ ID NO:73, SEQ ID NO:74, SEQ ID NO:76 and a nucleic acid sequence encoding an amino acid sequence selected from the group consisting of SEQ ID NO:78 and SEQ ID NO:87.

5. An assay kit for testing if an animal is susceptible to or has allergic dermatitis, said kit comprising:
(a) a formulation comprising at least one isolated ectoparasite saliva protein, wherein said ectoparasite saliva protein comprises an amino acid sequence selected from the group consisting of SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87; and
(b) a means for determining if said animal is susceptible to or has allergic dermatitis, wherein said means comprises use of said formulation to identify animals susceptible to or having allergic dermatitis.

6. A method to identify an animal susceptible to or having allergic dermatitis, said method comprising:
(a) administering to a site on said animal a formulation comprising at least one isolated ectoparasite saliva protein, wherein said ectoparasite saliva protein comprises an amino acid sequence selected from the group consisting of SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87; and
(b) comparing a reaction resulting from administration of said formulation with a reaction resulting from administration of a control solution, wherein said animal is determined to be susceptible to or to have allergic dermatitis if said reaction to said formulation is at least as large as said reaction to a positive control solution, and wherein said animal is determined not to be susceptible to or not to have allergic dermatitis if said reaction to said formulation is about the same size as said reaction to a negative control solution.

7. A method to identify an animal susceptible to or having allergic dermatitis by measuring the presence of antibodies indicative of allergic dermatitis in said animal, said method comprising:
(a) contacting a formulation with a body fluid from said animal under conditions sufficient for formation of an immunocomplex between said formulation and said antibodies, if present, in said body fluid, said formulation comprising at least one isolated ectoparasite saliva protein, wherein said ectoparasite saliva protein comprises an amino acid sequence selected from the group consisting of SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87; and
(b) determining the amount of immunocomplex formed, wherein formation of said immunocomplex indicates that said animal is susceptible to or has allergic dermatitis.

8. A method to desensitize a host animal to allergic dermatitis, comprising administering to said animal a therapeutic composition comprising a formulation comprising at least one isolated ectoparasite saliva protein, wherein said ectoparasite saliva protein comprises an amino acid sequence selected from the group consisting of SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87.

9. A method for prescribing treatment for allergic dermatitis, comprising:
(a) identifying an animal that is susceptible to or has allergic dermatitis by an in vivo or in vitro assay comprising a formulation comprising at least one isolated ectoparasite saliva protein, wherein said ectoparasite saliva protein comprises an amino acid sequence selected from the group consisting of SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87; and
(b) prescribing a treatment comprising administering said formulation to said animal.

10. The invention of claim 1 or 2, wherein said nucleic acid molecule comprises a nucleic acid sequence that encodes a flea saliva protein.

11. The invention of claim 1 or 2, wherein said nucleic acid molecule is a flea nucleic acid molecule.

12. The invention of claim 1 or 2, wherein said nucleic acid molecule is selected from the group consisting of Ctenocephalides, Ceratophyllus, Diamanus, Echidnophaga, Nosopsyllus, Pulex, Tunga, Oropsylla, Orchopeus and Xenopsylla nucleic acid molecules.

13. The invention of claim 1 or 2, wherein said nucleic acid molecule is selected from the group consisting of Ctenocephalides felis, Ctenocephalides canis, Ceratophyllus pulicidae, Pulex irritans, Oropsylla (Thrassis) bacchi, Oropsylla (Diamanus) montana, Orchopeus howardi, Xenopsylla cheopis and Pulex simulans nucleic acid molecules.

14. The invention of claim 1 or 2, wherein said nucleic acid molecule comprises a Ctenocephalides felis nucleic acid molecule.

15. The invention of claim 1, wherein said nucleic acid molecule hybridizes under stringent hybridization conditions with a nucleic acid molecule selected from the group consisting of nfspG5₅₉₅, nfspG5₂₇₀, nfspG5₂₁₃, nfspI₁₀₀₇, nfspN5₁₂₀₅, nf spN5₁₀₅₉, nfspN6₄₀₆ and nfspJ₄₂₀.

16. The invention of claim 1, wherein said nucleic acid molecule comprises a nucleic acid molecule selected from the group consisting of nfspG5₅₉₅, nfspG5₂₇₀, nfspG5₂₁₃, nfspI₁₀₀₇, nfspN5₁₂₀₅, nfspN5₁₀₅₉, nfspN6₄₀₆ and nfspJ₄₂₀.

17. The invention of claim 1 or 2, wherein said nucleic acid molecule is selected from the group consisting of: a nucleic acid molecule comprising a nucleic acid sequence that encodes a protein having an amino acid sequence selected from the group consisting of SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87; and a nucleic acid molecule comprising an allelic variant of a nucleic acid molecule encoding any of said amino acid sequences.

18. The invention of claim 1 or 2, wherein said nucleic acid molecule is selected from the group consisting of a nucleic acid molecule comprising a nucleic acid sequence selected from the group consisting of SEQ ID NO:52, SEQ ID NO:54, SEQ ID NO:55, SEQ ID NO:57, SEQ ID NO:58, SEQ ID NO:60, SEQ ID NO:61, SEQ ID NO:63, SEQ ID NO:64, SEQ ID NO:66, SEQ ID NO:67, SEQ ID NO:69, SEQ ID NO:71, SEQ ID NO:73, SEQ ID NO:74, SEQ ID NO:76 and a nucleic acid sequence encoding an amino acid sequence selected from the group consisting of SEQ ID NO:78 and SEQ ID NO:87.; and a nucleic acid molecule comprising an allelic variant of a nucleic acid molecule having any of said nucleic acid sequences.

19. The invention of claim 1 or 2, wherein said nucleic acid molecule comprises an oligonucleotide.

20. A recombinant molecule comprising a nucleic acid molecule as set forth in claim 1 or 2 operatively linked to a transcription control sequence.

21. A recombinant virus comprising a nucleic acid molecule as set forth in claim 1 or 2.

22. A recombinant cell comprising a nucleic acid molecule as set forth in claim 1 or 2, said cell being capable of expressing said nucleic acid molecule.

23. The invention of claim 3, wherein said protein, when administered to an animal, is capable of eliciting an immune response against a flea saliva protein.

24. The invention of claim 3, wherein said protein is selected from the group consisting of: a protein comprising an amino acid sequence selected from the group consisting of SEQ ID NO:53, SEQ ID NO:62, SEQ ID NO:65, SEQ ID NO:70, SEQ ID NO:72, SEQ ID NO:75, SEQ ID NO:77, SEQ ID NO:78 and SEQ ID NO:87; and a protein encoded by an allelic variant of a nucleic acid molecule encoding a protein comprising any of said amino acid sequences.

25. An isolated antibody that selectively binds to a protein as set forth in claim 3.

26. The invention of claim 4 or 5, wherein said allergic dermatitis is selected from the group consisting of flea allergy dermatitis, mosquito allergy dermatitis and Culicoides allergy dermatitis.

27. The invention of claim 4 or 5, wherein said allergic dermatitis comprises flea allergy dermatitis.

28. The invention of claim 4 or 8, wherein said composition further comprises at least one component selected from the group consisting of an excipient, an adjuvant and a carrier.

29. The invention of claim 4, wherein said composition comprises a controlled release composition.

30. The invention of claim 5, wherein said means of determining is selected from the group consisting of in vivo tests and in vitro tests.

31. The invention of claim 30, wherein said in vivo test comprises a skin test comprising:
(a) administering to a site on said animal said formulation and administering to a different site on said animal a control solution selected from the group consisting of positive control solutions and negative control solutions; and
(b) comparing a reaction resulting from administration of said formulation with a reaction resulting from administration of said control solution, wherein said animal is determined to be susceptible to or to have allergic dermatitis if said reaction to said formulation is at least as large as said reaction to said positive control solution, and wherein said animal is determined not to be susceptible to or not to have allergic dermatitis if said reaction to said formulation is about the same size as said reaction to said negative control solution.

32. The invention of claim 5 or 6, wherein said invention detects hypersensitivity selected from the group consisting of immediate hypersensitivity and delayed hypersensitivity.

33. The invention of claim 6 or 31, wherein said reaction is selected from the group consisting of a wheal, induration, erythema, and combinations thereof.

34. The invention of claim 6 or 31, wherein said positive control comprises histamine and said negative control comprises saline.

35. The invention of claim 30, wherein said in vitro test comprises a method for measuring the presence of antibodies indicative of allergic dermatitis in said animal, said method comprising:
(a) contacting said formulation with a body fluid from said animal under conditions sufficient for formation of an immunocomplex between said formulation and said antibodies, if present, in said body fluid; and
(b) determining the amount of immunocomplex formed, wherein formation of said immunocomplex indicates that said animal is susceptible to or has allergic dermatitis.

36. The invention of claim 5 or 7, wherein said formulation is immobilized on a substrate.

37. The invention of claim 7 or 35, wherein said antibodies comprise immunoglobulin IgE antibodies.

38. The invention of claim 5 or 7, wherein said invention detects immediate hypersensitivity in said animal.

39. The invention of claim 6, wherein said reaction is measured about 15 minutes after administration of said formulation to determine immediate hypersensitivity of said animal to said formulation.

40. The invention of claim 6, wherein said reaction is measured about 24 hours after administration of said formulation to determine delayed hypersensitivity of said animal to said formulation.

41. The invention of claim 7, wherein said body fluid is pretreated to remove non-IgE antibodies from said fluid.

42. The invention of claim 9, wherein said nucleic acid molecule is capable of hybridizing under stringent conditions with a nucleic acid sequence selected from the group consisting of SEQ ID NO:52, SEQ ID NO:54, SEQ ID NO:55, SEQ ID NO:57, SEQ ID NO:58, SEQ ID NO:60, SEQ ID NO:61, SEQ ID NO:63, SEQ ID NO:64, SEQ ID NO:66, SEQ ID NO:67, SEQ ID NO:69, SEQ ID NO:71, SEQ ID NO:73, SEQ ID NO:74, SEQ ID NO:76 and a nucleic acid sequence encoding an amino acid sequence selected from the group consisting of SEQ ID NO:78 and SEQ ID NO:87.
